(19) United States
(12) Patent Application Publication
(10) Pub. No.: US 2026/0102360 A1
(43) Pub. Date: Apr. 16, 2026

(54) NANO2 DODECAFLUOROPENTANE EMULSION AS A CARDIAC ARREST THERAPEUTIC

(71) Applicant: University of Florida Research Foundation, Incorporated, Gainesville, FL (US)

(72) Inventors: Torben K. BECKER, Gainesville, FL (US); Travis W. MURPHY, Miami, FL (US); Bruce Davis SPIESS, Palm Springs, CA (US)

(21) Appl. No.: 19/113,821

(22) PCT Filed: Oct. 16, 2023
(86) PCT No.: PCT/US2023/076993

§ 371 Date: Mar. 20, 2025

Related U.S. Application Data
(60) Provisional application No. 63/379,869, filed on Oct. 17, 2022.

(51) Int. Cl. A61K 31/02; A61K 9/00; A61K 9/107; A61P 9/04

(52) U.S. Cl. CPC A61K 31/02; A61K 9/0019; A61K 9/107; A61P 9/04

(57) ABSTRACT

The present invention provides methods for treating cardiac arrest in a subject. The methods involve administering to C1 the subject a pharmaceutical composition comprising NANO2 dodecafluoropentane emulsion.

### CROSS REFERENCE TO RELATED APPLICATION

This application claims the benefit under 35 USC 119(e) of U.S. Provisional Application No. 63/379,869, filed Oct. 17, 2022, which is incorporated by reference in its entirety for all purposes.

### BACKGROUND OF THE INVENTION

Cardiac arrest is the final stage of all forms of shock immediately preceding death, and a major public health problem. Cardiac arrest affects more than 550,000 Americans every year, but survival rates remain low, at about 10-15% [3]. Rates for survival without severe neurological injury remain even lower, at about 8%. Anoxic brain injury is irreversible and becomes severe after only 5 minutes [2]. Anoxic brain injury and its sequelae are the limiting factors in improving the outcomes of cardiac arrest patients. Over the last decade, improvements in coronary care, such as lay-person resuscitation, rapid emergency medical services (EMS) response, public access defibrillation, and a focus on simplifying first aid interventions, have led to significant gains in successful cardiac resuscitation, however, severe brain injury due to lack of oxygen remains as the rate-limiting step towards improved survival from cardiac arrest [3-6].

Despite advances in cardiac arrest care, survival rates remain low, and survival without severe neurological injury even lower [1]. Anoxic brain injury is irreversible and becomes severe after 5 minutes of malperfusion [2]. Efforts to improve cardiac arrest care through bystander and emergency medical service initiatives, public access defibrillation, targeted temperature management, and mechanical circulatory support have shown promise, though anoxic injury to the brain and other organs remains a rate-limiting step in improving functional survival.[3-6].

Perfluorocarbon emulsions (PFC) have unique characteristics including enhanced gas solubility that improve oxygen delivery in low- and no-flow scenarios by increasing oxygen diffusibility. Studies of PFC for cardiac arrest and with coronary occlusion have been performed as a means for inducing hypothermia using total liquid ventilation and isolated organ perfusion, both impractical in out-of-hospital cardiac arrest (OHCA)[7-11]. While several models of single-organ injury have been studied with intravenous PFC, therapeutic administration for the hypoperfusion induced by cardiac arrest has yet to be explored [7, 12-24].

### BRIEF SUMMARY OF THE CLAIMED INVENTION

In one aspect, the invention provides a method of treating a subject with cardiac arrest, comprising administering to the subject a pharmaceutical composition comprising NANO2 dodecafluoropentane emulsion (DDFPe) (NuvOx Pharma, LLC, Tucson, Arizona). In some methods, the cardiac arrest is associated with coronary artery disease, valvular heart disease, cardiac dysrhythmias, myocardial infarction, pulmonary disease, arrhythmia, stroke, shock, sepsis, pneumonia, pulmonary embolism, trauma, or COVID-19. In some methods, blood flow has stopped in the subject.

In some methods, the pharmaceutical composition is administered intravenously. In some methods, the pharmaceutical composition is injected intravenously via bolus over about 1 to about 10 minutes at a dose of about 0.3 mL/kg of subject body weight to about 1 mL/kg of subject body weight. In some methods, the pharmaceutical composition is injected intravenously via slow IV push over about 1 to about 15 minutes at a dose of about 0.3 mL/kg to about 1 mL/kg of subject body weight. In some methods, the pharmaceutical composition is injected intravenously via slow IV push over about 1 to about 10 minutes. In some methods, the pharmaceutical composition is injected intravenously via slow IV push over about 5 to about 15 minutes.

In some methods, the pharmaceutical composition is injected intravenously at a dose of about 0.5 mL/kg of subject body weight. In some methods, the pharmaceutical composition is injected intravenously over about 10 minutes at a dose of about 0.5 mL/kg of subject body weight.

In some methods, the pharmaceutical composition is administered as an IV infusion at a rate of about 0.05 mL/kg of subject body weight per hour to about 0.15 mL/kg of subject body weight per hour. In some methods, the pharmaceutical composition is injected intravenously as a sustained IV infusion at a rate of about 0.001 mL/kg of subject body weight per hour to about 0.015 mL/kg of subject body weight per hour.

In some methods, the subject is a mammal. For example, the mammal can be a primate. For example, the mammal can be a non-human primate. For example, the mammal can be a human. For example, the mammal can be a rodent. For example, the rodent can be a mouse, rat, guinea pig, hamster, or gerbil. In some methods, the mammal is selected from the group consisting of human, baboon, chimpanzee, monkey, cynomolgus, marmoset, rhesus, rodent, rabbit, cat, dog, horse, cow, sheep, goat, pig, ferret, guinea pig, hamster, and gerbil.

A further understanding of the nature and advantages of the present invention may be realized by reference to the remaining portions of the specification and claims.

### BRIEF DESCRIPTION OF THE DRAWINGS

FIG. 1 depicts histopathology images of hematoxylin and eosin (H&E) stained brain tissue samples from placebo-treated animal #3 and DDFPe-treated animals #4 and #5. 1A: Pig #4 parietal cortex; 1B: Pig #5 parietal cortex; 1C: Pig #3 parietal cortex 1D: Pig #4 dorsal hippocampus; 1E: Pig #5 dorsal hippocampus; 1F: Pig #3 dorsal hippocampus. 1A and 1D. Near normal appearance in parietal cortex area (1A) and dorsal hippocampus (1D), scale −/+; 1B and 1E. Ischemic damage showing some degenerated neurons with moderate edema (vacuolization) in parietal cortex (1B) and dorsal hippocampus (IE), scale +˜++; 1C and 1F. More damage to neurons and edematous areas in parietal cortex (1C) and dorsal hippocampus (1F) scaled as ++˜+++.

FIG. 2 depicts GFAP immunohistochemical stained images of brain tissue samples from placebo-treated animal #3 and DDFPe-treated animals #4 and #5. 2A: Pig #4; 2B: Pig #5; 2C: Pig #3. GFAP immunohistochemistry shows activation of astrocytes after cardiac arrest. 2A. No or a few activated astrocytes, 2B. Moderate activation of astrocytes, and 2C. Activation of astrocytes.(40X).

FIG. 3 depicts time to sustained normal functional status for each animal, where time is hours post-ROSC. x-axis: time (hours), y-axis (pig number) * denotes placebo-treated animal.

FIGS. 4A-4C depict Box-and-Whisker Plots Showing Aggregate Progression of Functional Scores. 4A (top plot): Overall Performance Category (OPC) values; 4B (middle plot): Neurological Alertness Score (NAS) values; 4C (bottom plot): Neurological Dysfunction Score (NDS) values, at 24 hr post-ROSC, 48 hr post ROSC, 72 hr post-ROSC, and 96 hr post-ROSC. Solid-filled (placebo); Dotted-filled (DDFPe).

FIG. 5 is a table depicting Neuronal Damage Scale (H&E staining)) and GFAP staining scale
*Pig #3 received placebo
−/+ near normal, a few damages, no significant activation of astrocytes (GFAP); mild damage
+/++ mild ˜moderate neuronal damage and activated astrocytes (GFAP)
++/+++ many damaged neurons and many spot of activated astrocytes (GFAP)

FIG. 6 is a table depicting 24 hr Interval Functional Scoring for All Subjects
* Indicates no record due to subject not surviving experimental cardiac arrest
Neurologic Scoring included Overall Performance Score (OPC), Neurological Alertness Score (NAS), Neurological Dysfunction Score (NDS)

### Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1st ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1′ ed., 1999); Dictionary of Pharmaceutical Medicine, Nahler (Ed.), Springer-Verlag Telos (1994); Dictionary of Organic Chemistry, Kumar and Anand (Eds.), Anmol Publications Pvt. Ltd. (2002); and A Dictionary of Biology (Oxford Paperback Reference), Martin and Hine (Eds.), Oxford University Press (4^{th} ed., 2000). In addition, the following definitions are provided to assist the reader in the practice of the invention.

The term “patient” includes human and other mammalian subjects that receive either prophylactic or therapeutic treatment.

The term “biological sample” refers to a sample of biological material within or obtainable from a biological source, for example a human or mammalian subject. Such samples can be organs, organelles, tissues, sections of tissues, bodily fluids, peripheral blood, blood plasma, blood serum, cells, molecules such as proteins and peptides, and any parts or combinations derived therefrom. The term biological sample can also encompass any material derived by processing the sample. Derived material can include cells or their progeny. Processing of the biological sample may involve one or more of filtration, distillation, extraction, concentration, fixation, inactivation of interfering components, and the like.

The term “disease” refers to any abnormal condition that impairs physiological function. The term is used broadly to encompass any disorder, illness, abnormality, pathology, sickness, condition, or syndrome in which physiological function is impaired, irrespective of the nature of the etiology.

The term “symptom” refers to a subjective evidence of a disease, as perceived by the subject. A “sign” refers to objective evidence of a disease as observed by a physician.

The term “individual” or “subject” refers to any mammal, including any animal classified as such, including humans, non-human primates, primates, baboons, chimpanzees, monkeys, cynomolgus, marmoset, rhesus, rodents (e.g., mice, rats), rabbits, cats, dogs, horses, cows, sheep, goats, pigs, ferrets, guinea pigs, hamsters, gerbils etc.

Examples of a condition associated with cardiac arrest are coronary artery disease, valvular heart disease, cardiac dysrhythmias, myocardial infarction, pulmonary disease, arrhythmia, stroke, shock, sepsis, pneumonia, pulmonary embolism, trauma, and COVID-19 complications. Cardiac arrest is also associated with permanent brain disorders such as confusion, neuropsychologic dysfunction, inability to care for oneself, reduction in learning, reduction in ability to be employed and/or reduction in ability to do activities of daily living. Profound neurologic disorders and disability are common after even short cardiac arrest periods.

The terms “therapeutically effective dose,” or “therapeutically effective amount,” refer to that amount of a compound that results in prevention, delay of onset of symptoms, or amelioration of symptoms of cardiac arrest. A therapeutically effective amount will, for example, be sufficient to treat, prevent, reduce the severity, delay the onset, or reduce the risk of occurrence of one or more symptoms of cardiac arrest. The effective amount can be determined by methods well known in the art and as described in subsequent sections of this description.

The terms “treatment,” “therapeutic method,” and their cognates refer to treatment and prophylactic/preventative measures. Those in need of treatment may include individuals already having a particular medical disorder as well as those who may ultimately acquire the disorder. The need for treatment is assessed, for example, by the presence of one or more risk factors associated with the development of a disorder, the presence or progression of a disorder, or likely receptiveness to treatment of a subject having the disorder. Treatment may include slowing or reversing the progression of a disorder.

The term “intravenous injection” (IV) refers to any method of actively injecting a substance, for example a medication, or allowing it to infuse by gravity feed into a vein of the circulation, for example a peripheral vein or a large vessel of the central venous circulation. The term “bolus injection” refers to a rapid injection of a defined amount of substance, for example a medication, either by weight (for example, mg or g) or by volume amount. The term “slow push injection” refers to a slow IV injection by manual compression of a plunger on a syringe thereby pushing a substance, for example a medication, into a vein, often providing an approximately equal amount per minute. The term “IV infusion” refers to administration of a substance, for example a medication, via an infusion pump (for example a programmed medication pump) or via gravity (in the latter case, usually using a drip infusion set) over an interval of time (for example, minutes to hours). The term “sustained IV infusion” refers to IV infusion that is being administered over a longer duration of time, such as several hours or days.

Compositions or methods “comprising” or “including” one or more recited elements may include other elements not specifically recited. For example, a composition that “comprises” or “includes” NANO2 DDFPe may contain NANO2 DDFPe alone or in combination with other ingredients. When the disclosure refers to a feature comprising specified elements, the disclosure should alternative be understood as referring to the feature consisting essentially of or consisting of the specified elements. Moreover, elements that are shown or described as being combined with other elements, can, in various embodiments, exist as stand-alone elements.

Designation of a range of values includes all integers within or defining the range, and all subranges defined by integers within the range.

Unless otherwise apparent from the context, the term “about” encompasses insubstantial variations, such as values within a standard margin of error of measurement (e.g., SEM) of a stated value. Unless otherwise apparent from the context, the term “about” encompasses values within ±5% or ±10% of a stated value.

Statistical significance means p 0.05.

The singular forms of the articles “a,” “an,” and “the” include plural references unless the context clearly dictates otherwise. For example, the term “a compound” or “at least one compound” can include a plurality of compounds, including mixtures thereof.

### DETAILED DESCRIPTION

### I. General

The present invention provides methods of treating cardiac arrest in a subject using NANO2 dodecafluoropentane emulsion (DDFPe) (NuvOx Pharma, LLC, Tucson, Arizona). The methods are useful in improving brain oxygenation during cardiac arrest, in preventing additional hypoxemic injury, and/or in reducing the severity of anoxic injury. The methods are useful in treating cardiac arrest in humans and animals.

For example, the present invention provides a method of using NANO2 DDFPe as a therapeutic agent in cardiac arrest in a swine model. Among 12 animals, 7 of which who received NANO2 DDFPe, the return-of-spontaneous circulation and the survival rate were twice the rate of the animals who received saline placebo. Establishing this model is significant because the oxygen-carrying and oxygen-unloading effects of NANO2 DDFPe are hypothesized to improve brain oxygenation during cardiac arrest, thus preventing additional hypoxemic injury and reducing the severity of anoxic injury.

### II. DDFPe

Dodecafluoropentane emulsion (DDFPe) is a short half-life PFC with very high oxygen solubility and has been shown to be safe for intravenous administration.[10,21, 25-27] DDFPe can oxygenate even with microcirculatory compromise in animal models of vascular occlusion or hemorrhage [20, 22, 23, 27, 28]. Moreover, DDFPe's benefits in human stroke patients provide an evidentiary basis supporting a possible therapeutic effect on hypoxic-ischemic brain injury and the sequelae of impaired microcirculation in cardiac arrest [13,16,20-22, 24].

Intravenous NANO2 DDFPe (NuvOx Pharma, LLC, Tucson, Arizona) has been shown to have neuroprotective effects in acute ischemic stroke and beneficial outcomes in other ischemic events in various animal models, including swine, rat, and rabbit, and in human [16]. The oxygen-transporting nanodroplet DDFPe, given intravenously in the first 3 hours, can reduce stroke symptoms and stroke volumes by more than 80% and appears to widen the window for therapy by many hours ([20, 51]. The apparent mechanism is improved oxygen transport by DDFPe nanodroplets (250 nanometers in diameter) into ischemic zones where oxygen transport by erythrocytes (8 microns in diameter) is reduced. Although collaterals seem to provide the bulk of oxygen delivery to tissue distal to an occlusion [52], the droplets may be able to pass through blockages that prevent passage of erythrocytes, which are more than 30 times larger in diameter [21, 53]. Better DDFPe oxygenation may improve metabolism and reduce the edema that disrupts erythrocyte movement in capillaries. Improved blood flow may then add erythrocyte hemoglobin transport to the DDFPe oxygen transport ([16].

DDFPe is the first perfluorocarbon and oxygen therapeutic to enter a phase II randomized clinical trial in stroke. Safety of intravenous DDFPe has been demonstrated in animal studies. It transports 3-7 times more oxygen than prior longer half-lifed perfluorocarbons and 9-15 times more than blood. It is effective below levels expected to cause adverse events (AEs-toxicity levels) ([21, 54]. However, large doses given in quick repetition caused pulmonary edema in 1 study in dogs [55] and if the nanodroplets were heated or otherwise damaged, they may coalesce and cause pulmonary emboli. Prior safe use as an ultrasound contrast agent (for example under name EchoGen) in over 2200 patients involved an activated form of DDFPe subjected to brief negative pressure in a syringe before injection to promote micro-bubbles and increase visibility by ultrasound [25, 56, 57]. DDFPe is almost completely eliminated through the lungs as a gas, with a therapeutic span of about 2 hours per dose [21, 13, 25].

DDFP is also known as perfluoropentane. DDFPe and its preparation are described in the art. DDFPe is also known as perflenapent emulsion, EchoGen, NVX-108, NVX-408, and NANO2 DDFPe.

In an example, perflenapent emulsion (EchoGen) is a 2% aqueous emulsion of dodecafluoropentane (DDFP) stabilized by a surfactant in a 30% sucrose solution [58].

In an example, EchoGen emulsion (Perflenapent Emulsion) is 1) dispersed phase: dodecafluoropentane, 2% w/v; 2) stabilizer; fluorosurfactant; and 3) continuous phase: sucrose, 30% w/v [59].

Under name EchoGen and provided by Sonus Pharmaceuticals Ltd., DDFPe was approved as an ultrasound contrast agent [60]. In an example, EchoGen is provided as a 2% w/v emulsion for injection, and each 1 ml emulsion of for injection contains 20 mg of a mix of perflenapent (approximately 82%) and perflisopent (approximately 18%). Perflenapent and perflisopent are the n-pentane and iso-pentane structural isomers of dodecafluoropentane, respectively. The active substance in EchoGen is dodecafluoropentane in the form of a liquid/liquid emulsion stabilized by a surfactant (PEG Telomer B) [56].

DDFPe and its preparation are described in U.S. Pat. No. 5,876,696 (e.g., in Example 44 of U.S. Pat. No. 5,876,696, column 27, line 65 through column 45, line 18). In an example, DDFPe is prepared using the following equipment and steps: Microfluidizer, Model 110Y, Interaction chamber pressure 14,000 PSI; Pressure vessels, 316 steel, 5 L and 12 L sizes; Filters, cellulose acetate, 0.22 micron; Filter holders, 142 mm. The following solutions are made: 33.3% (w/v) sucrose, 20 L; 150.0 g Pluronic P-123, 150.0 g Zonyl FSO, 2.5 L, sonicate to aid dissolution (stock surfactant solution). The Microfluidizer is primed with the sucrose solution. The interaction chamber, tubing, and cooling coil are covered with chipped ice during the comminution process. To a 5 L pressure vessel with stir bar in an ice bath add sequentially: 1800 mL stock surfactant solution; 333 g dodecafluoropentane. Pressurize vessel to 10 PSI with nitrogen for 60 min while stirring. Pass the suspension through the Microfluidizer at 14,000 PSI for 160 min and with a circulating water bath cooling the interaction chamber to −3.0° C. Transfer the emulsion to a vessel containing 18 L of 33.3%, w/v, sucrose at 4° C. and mix for 45 min. Transfer the emulsion to 20 mL prechilled vials using positive pressure, passing the material through a 0.22 micron filter in the process. Cap and seal the vials.

In an example, a DDFPe formulation is as in [61], where the formulation is a 2% emulsion of dodecafluoropentane (DDFP) in sucrose-containing saline.

In an example, a DDFPe formulation is as in [62], where an aqueous nano-emulsion of DDFP is used, wherein the intravenous injectable contains 0.3% weight/volume (w/v), PEG telomer-B, 0.3% w/v Pluronic P123 and 30% w/v sucrose in addition to 2% DDFP w/v.

In an example, a DDFPe formulation is prepared as in [21] or as in US Patent Publication US 2018/0069389 A1 (paragraph [0095]), where for each 1 liter batch, 3 grams of PEG-Telomer B and 20 grams of DDFP are homogenized along with a 33% sucrose solution using a custom-build semi-enclosed stainless steel containment system attached to an Avestin Emulsiflex-C5 homogenizer, and wherein each homogenate is processed for 6 passes through the chamber at 14, 000 psi and then terminally sterile filtered immediately prior to filling into 5 mL vials, and wherein the vials are stoppered and crimped and then stored at room temperature.

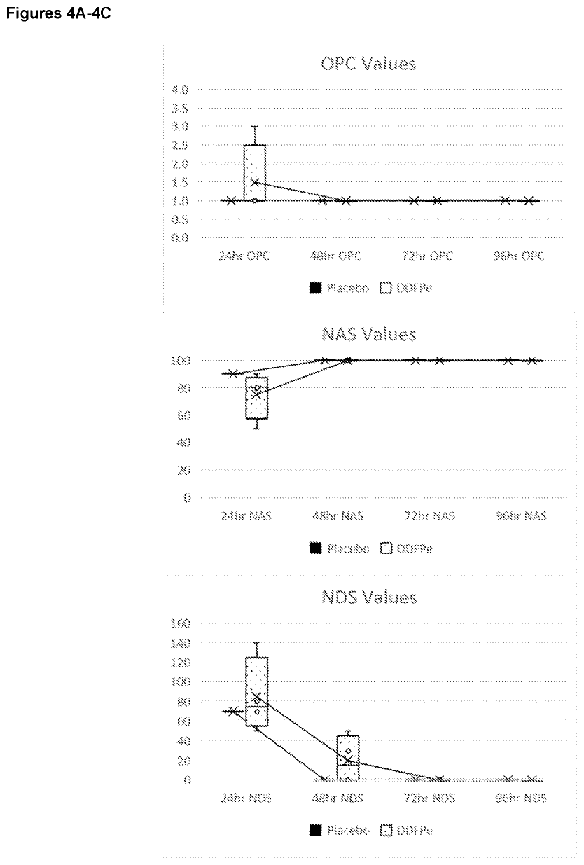

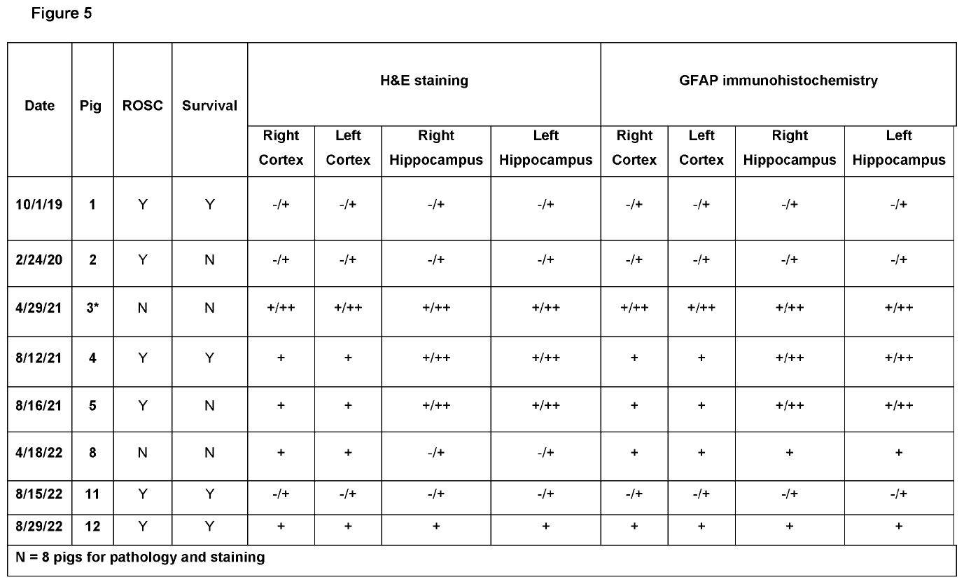

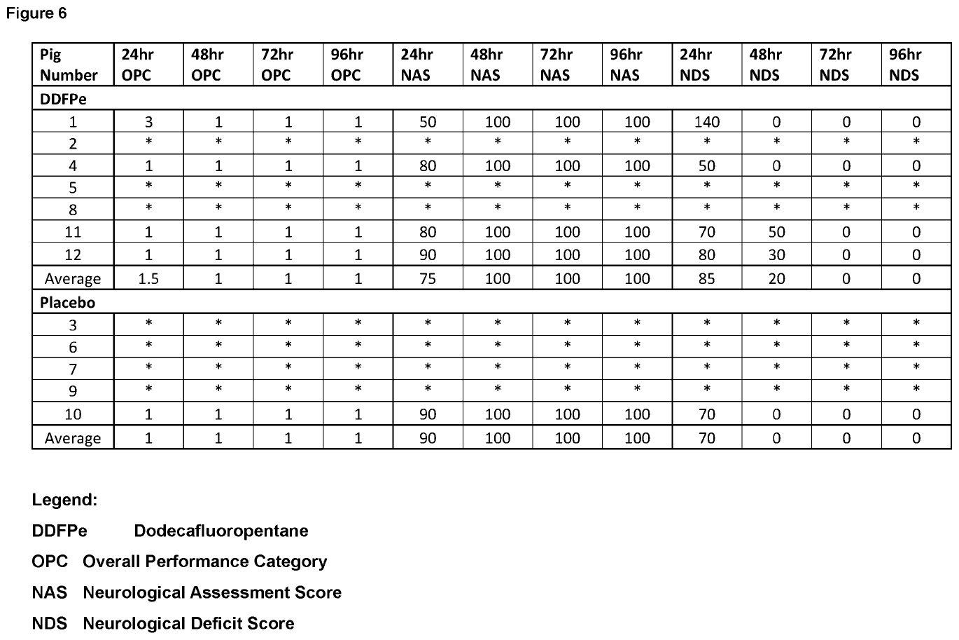

Methods of the present invention may use buffered or unbuffered DDFPe. Buffered DDFPe is described for example in U.S. Pat. No. 5,876,696 (e.g., at column 3, lines 23-35 and column 10, lines 47-56). An exemplary buffered form of DDFPE, where DDFPe is buffered with a phosphate buffer, for example a 0.01 M phosphate buffer, is described in U.S. Pat. No. 8,822,549 (for example, see Examples 1-3 of U.S. Pat. No. 8,822,549). An exemplary buffered form of DDFPe (NVX-108) buffered at physiological pH (˜7) is described in [63]. An exemplary buffered form of DDFPe (NVX-108) is described in US Patent Publication US 2018/0221302 A1 (e.g., at FIG. 4 and paragraph [0033]) as a nanoemulsion of perfluoropentane (2% w/v) and PEG-Telomer B surfactant (0.3% w/v) in phosphate buffered 30% aqueous sucrose solution. An exemplary buffered form of DDFPe (2% emulsion) is described [64] as NVX-428, and the active component of NVX-428 is dodecafluoropentane (DDFP<2% w/v), where DDFP is stabilized into an emulsion (DDFPe=NVX-428) with surfactant in a buffered sucrose solution and wherein the formula is sonicated.

### III. Use of DDFPE as a Therapeutic for Cardiac Arrest

The studies in the Examples evaluated the therapeutic role of dodecafluoropentane emulsion (DDFPe) in the setting of cardiac arrest and its impact on functional neurologic outcome.

The purpose of the studies was to establish the potential therapeutic role of DDFPe in the mitigation and treatment of hypoperfusion secondary to cardiac arrest—with improved odds of resuscitation and functional recovery the primary goals.

This swine model is a first-of-its-kind investigation of intravenous perfluorocarbon emulsion for cardiac arrest treatment. The findings of the studies in the Examples suggest that there is a potential role for DDFPe as a therapeutic intervention to limit the extent of hypoxic-ischemic injury and increase functionally-intact survival after cardiac arrest[7, 12-24].

The trend towards improved ROSC rate and functionally intact survival are thought to reflect improved microcirculatory oxygen delivery during the low- and no-flow states of cardiac arrest. Several recent studies have established microthrombi's role in the small capillary beds as culprits for decreased tissue oxygenation during low-flow states like CPR (cardiopulmonary resuscitation) and even once macrovascular blood flow is restored following ROSC [42-44]. Oxygen transport characteristics of PFCs like DDFPe are different than the chemical binding of oxygen to hemoglobin as PFCs allow for oxygen delivery by chains of PFC microparticles [45-46, 21]. Oxygen is dissolved in PFC particles and moves by diffusion down a gradient from high concentrations (such as erythrocytes) to areas with high tissue demand [46, 21]. In cardiac arrest, convective forward flow of microcirculatory erythrocytes is essentially halted [42-44, 47]. Therefore, a plasma additive that enhances diffusivity of oxygen such as PFC could enhance critical oxygen delivery [11, 46, 21]. Due to DDFPe's less than 0.2 m diameter, emulsion particles can diffuse to ischemic tissue via collateral perfusion or even via small or nearly-blocked capillaries where erythrocytes cannot physically fit [10,21,48]. Even in models of vascular occlusion, oxygen delivery occurs if PFC is present [13, 20, 23, 28]. This ability to reduce ischemic-hypoxic injury has been demonstrated in several single-organ injury models and human trials where PFCs have demonstrated decreased ischemic-hypoxic complications and increased oxygenation and tissue viability [7, 12-24].

Histologic and biomarker evidence of injury in areas of the brain most sensitive to cardiac arrest confirms that the animals in our model sustained significant neurological injury prior to the administration of the study drug, creating the environment necessary to detect potential therapeutic benefit. Significant potential for clinical benefit is suggested as more pigs in the intervention arm were able to achieve ROSC and return to normal functional status. Additionally, increased ROSC in the DDFPe group resulted in lessened need for repeated defibrillations, ongoing chest compressions or vasoactive epinephrine. This finding is compelling as severe hypoxic-ischemic brain injury is exacerbated by microcirculatory dysfunction and remains the rate-limiting step towards improved cardiac arrest survival [43,44]. The larger NFL (neurofilament light chain) increase over the 96-hour observation seen in the DDFPe group can be attributed to the higher survival rate, providing an opportunity for ongoing sampling as opposed to placebo animals that did not survive experimental arrest.

Additionally, murine coronary occlusion studies found beneficial effects of DDFPe extending beyond the previously reported 2-minute half-life due to suspected delay of hypoxic injury and induction of ischemic-preconditioning as DDFPe was cleared [15,28]. In healthy human volunteers, the terminal half-life for DDFPe was shown to be 120 minutes [25].

As with any new therapy, some concerns may arise about the long-term effects of PFCs like DDFPe, and species-specific complications such as pulmonary hypertension and activation of macrophages have been seen in pigs [7]. However, prior work including measures of vital signs, ECG, blood oximetry, serum, urine, and coagulation analytes, spirometry, complement activation assay, echocardiography, physical exam, discomfort evaluation, and adverse events has shown administration of nonoxygenated DDFPe to healthy human males at doses up to 0.35 mL/kg to be safe with an elimination half-life of unchanged drug in exhaled gas complete by 120 minutes [25,26,49]. Overall, this risk profile should be considered acceptable, and the potential benefits compelling for further work using PFCs such as DDFPe in cardiac arrest.

### Limitations

Firstly, the small sample size of the present study is acknowledged. While it is difficult to draw definitive conclusions, the combination of clinical and functional findings suggests a potential benefit that larger studies may define more precisely. A single-sex pig was used, due to prior institutional experience in keeping with similar studies, but as pigs were prepubertal, any contribution of estrogen to survival is expected to be minimal [7, 29, 50].

The presence of organ injuries after prolonged CPR is a well-known complication, especially with mechanical chest compression devices, and may have negated a clinical benefit from DDFPe in animals that did not survive the experimental arrest. Due to an inability to provide ongoing critical care as would be provided in humans, these animals required euthanasia.

### CONCLUSIONS

Cardiac arrest is the final stage of shock, with neurologically intact survival less than 10%. Improving microcirculatory oxygen delivery to diminish the impact of hypoperfusion may provide significant therapeutic benefit. Echoing the findings of human studies for stroke, the findings of this study suggest a potential therapeutic role of PFCs like DDFPe in the treatment of cardiac arrest. Adequately powered preclinical studies are necessary to build the case for translational trials.

### IV. Methods of Using NANO2 DDFPe as a Therapeutic for Treating Cardiac Arrest

The methods herein can be used to treat a subject for cardiac arrest when blood flow has stopped or within about four hours of blood flow interruption, for example within about 15-30 minutes, within about 15 minutes, within about 30 minutes, within about 1 hour, within about 2 hours, within about 3 hours, or within about 4 hours of blood flow interruption. A route of administration for NANO2 DDFPe is intravenous (IV), for example via bolus, slow IV push or sustained infusion.

In some methods, NANO2 DDFPe is administered intravenously as a bolus over about 1 to about 10 minutes at a dose of about 0.3 mL/kg to about 1 mL/kg of subject body weight. In some methods, the dose is about 0.3 mL/kg, about 0.4 mL/kg, about 0.5 mL/kg, about 0.6 mL/kg, about 0.7 mL/kg, about 0.8 mL/kg, about 0.9 mL/kg, or about 1.0 mL/kg, of subject body weight. Some doses are 0.3 mL/kg, 0.4 mL/kg, 0.5 mL/kg, 0.6 mL/kg, 0.7 mL/kg, 0.8 mL/kg, 0.9 mL/kg, or 1.0 mL/kg, of subject body weight.

In some methods, NANO2 DDFPe is administered intravenously as a slow IV push over about 1 to about 15 minutes, for example about 1 to about 10 minutes or about or about 5 to about 15 minutes, at a dose of about 0.3 mL/kg to about 1 mL/kg of subject body weight. In some methods, the dose is about 0.3 mL/kg, about 0.4 mL/kg, about 0.5 mL/kg, about 0.6 mL/kg, about 0.7 mL/kg, about 0.8 mL/kg, about 0.9 mL/kg, or about 1.0 mL/kg, of subject body weight. In some methods, the dose is 0.3 mL/kg, 0.4 mL/kg, 0.5 mL/kg, 0.6 mL/kg, 0.7 mL/kg, 0.8 mL/kg, 0.9 mL/kg, or 1.0 mL/kg, of subject body weight.

In some methods, NANO2 DDFPe is injected intravenously over about 10 minutes at a dose of about 0.5 mL/kg of subject body weight. In some methods, NANO2 DDFPe is injected intravenously over 10 minutes at a dose of 0.5 mL/kg of subject body weight. In some methods, NANO2 DDFPe is injected intravenously at a dose of about 0.5 mL/kg of subject body weight. In some methods, NANO2 DDFPe is injected intravenously at a dose of 0.5 mL/kg of subject body weight. In an example, a swine of ˜40 kg body weight is administered 20 ml over 10 minutes, 0.5 ml per kg of weight of animal.

In some methods, NANO2 DDFPe is administered as an IV infusion, at a rate of about 0.05 mL/kg of subject body weight per hour to about 0.15 mL/kg of subject body weight per hour. In some methods, the IV infusion is at a rate of about 0.05 mL/kg of subject body weight per hour, about 0.06 mL/kg of subject body weight per hour, about 0.07 mL/kg of subject body weight per hour, about 0.08 mL/kg of subject body weight per hour, about 0.09 mL/kg of subject body weight per hour, about 0.1 mL/kg of subject body weight per hour, about 0.11 mL/kg of subject body weight per hour, about 0.12 mL/kg of subject body weight per hour, about 0.13 mL/kg of subject body weight per hour, about 0.14 mL/kg of subject body weight per hour, or about 0.15 mL/kg of subject body weight per hour. In some methods, the IV infusion is at a rate of 0.05 mL/kg of subject body weight per hour, 0.06 mL/kg of subject body weight per hour, 0.07 mL/kg of subject body weight per hour, 0.08 mL/kg of subject body weight per hour, 0.09 mL/kg of subject body weight per hour, 0.1 mL/kg of subject body weight per hour, 0.11 mL/kg of subject body weight per hour, 0.12 mL/kg of subject body weight per hour, 0.13 mL/kg of subject body weight per hour, 0.14 mL/kg of subject body weight per hour, or 0.15 mL/kg of subject body weight per hour.

In some methods, NANO2 DDFPe is injected intravenously as a sustained IV infusion at a rate of about 0.001 mL/kg of subject body weight per hour to about 0.015 mL/kg of subject body weight per hour. In some methods, the sustained IV infusion is at a rate of about 0.001 mL/kg of subject body weight per hour, about 0.002 mL/kg of subject body weight per hour, about 0.003 mL/kg of subject body weight per hour, about 0.004 mL/kg of subject body weight per hour, about 0.005 mL/kg of subject body weight per hour, about 0.006 mL/kg of subject body weight per hour, about 0.007 mL/kg of subject body weight per hour, about 0.008 mL/kg of subject body weight per hour, about 0.009 mL/kg of subject body weight per hour, about 0.01 mL/kg of subject body weight per hour, about 0.011 mL/kg of subject body weight per hour, about 0.012 mL/kg of subject body weight per hour, about 0.013 mL/kg of subject body weight per hour, about 0.014 mL/kg of subject body weight per hour, or about 0.015 mL/kg of subject body weight per hour. In some methods, the sustained IV infusion is at a rate of 0.001 mL/kg of subject body weight per hour, 0.002 mL/kg of subject body weight per hour, 0.003 mL/kg of subject body weight per hour, 0.004 mL/kg of subject body weight per hour, 0.005 mL/kg of subject body weight per hour, 0.006 mL/kg of subject body weight per hour, 0.007 mL/kg of subject body weight per hour, 0.008 mL/kg of subject body weight per hour, 0.009 mL/kg of subject body weight per hour, 0.01 mL/kg of subject body weight per hour, 0.011 mL/kg of subject body weight per hour, 0.012 mL/kg of subject body weight per hour, 0.013 mL/kg of subject body weight per hour, 0.014 mL/kg of subject body weight per hour, and 0.015 mL/kg of subject body weight per hour.

In some methods, the dose of NANO2 DDFPe is repeated as needed, for example from about 1 to about 50 times (e.g., from about 1 to about 25 times, from about 1 to about 10 times, from about 1 to about 5 times, from about 1 to about 3 times, from about 2 to about 10 times). The present regimen can be administered in combination with another a second therapeutic agent effective in treatment or prophylaxis of cardiac arrest before, during or after the administration of NANO2 DDFPe to the subject. The NANO2 DDFPe may be co-administered with one or more other suitable second therapeutic agents, or one or more such second therapeutic agents may be incorporated into the NANO2 DDFPe. Exemplary second therapeutic agents include, but not limited to adrenaline, atropine, amiodarone, lidocaine, sodium bicarbonate, and calcium.

In some methods, biomarkers of a subject may be measured to assess severity of brain damage in the subject. Brain damage is usually the rate limiting step for recovery from cardiac arrest. Biomarkers can be measured in a blood sample from a subject before, during, or after treatment with NANO2 DDFPe. Exemplary biomarkers measured are NSE (Neuron-specific enolase; neuronal marker), UCH-L1 (ubiquitin C-terminal hydrolase-L1; neuronal cell body marker), GFAP (glial fibrillary acidic protein; astroglial injury/gliosis marker), S100B (astroglial and blood-brain-barrier injury marker), NF-L (neurofilament light chain (NFL); axonal injury marker) and Tau (neurodegenerative marker). (e.g., SR-X Data Sheet Simoa N4PB Kit, Quanterix Corporation, Billerica, MA; [65]. In some methods, brain samples from a subject are collected before, during, or after treatment with NANO2 DDFPe and analyzed for severity and extent of ischemic neuronal change, infarcts, and edema.

In addition, all aforementioned embodiments are applicable to domesticated, agricultural, or zoo-maintained mammals experiencing cardiac arrest, as well as to humans. For example, NANO2 DDFPe may be administered to humans, non-human primates, primates, baboons, chimpanzees, monkeys, cynomolgus, marmoset, rhesus, rodents (e.g., mice, rats), rabbits, cats, dogs, horses, cows, sheep, goats, pigs, ferrets, guinea pigs, hamsters, gerbils etc. NANO2 DDFPe may be administered to house pets such as dogs, cats, rabbits, ferrets, guinea pigs, hamsters and gerbils, as well as to agricultural animals, such as horses, sheep, cows, and pigs, or to animals such as camel, cynomolgus, marmoset, rhesus and chimpanzee. NANO2 DDFPe may be administered to a human. NANO2 DDFPe may be administered to a pig.

### EXAMPLES

### Example 1: Use of NANO2 Dodecafluoropentane Emulsion in an Adult Swine Model of Cardiac Arrest

Ventricular fibrillation and thus cardiac arrest were induced in adult swine, adopted from a previously described animal model of cardiac arrest [38]. Animals were then resuscitated with the use of NANO2 DDFPe vs. placebo in addition to standard resuscitative care.

Physiologic markers of oxygen levels in the blood stream were monitored in tissues and brain during and after resuscitation. Once resuscitated, the animals' behavior was observed for 96 hours to further evaluate the effect of the NANO2 DDFPe on neurological injury and recovery though standardized observation and microscopic examination.

Standardized and validated scoring systems were used for animal observation, the neurological alertness score (NAS), the neurological dysfunction score (NDS) and overall performance score (OPC) [=neurological assessment panel][66, 30, 67, 31, 32, 34, 33]. Subsequently, the brain was examined on a microscopic level to assess and score the degree of cell injury [34, 33, 36]. Based on these data, a comprehensive profile of the effects of NANO2 DDFPe in cardiac arrest, that includes physiological, observational-behavioral and histological data points, was created.

### Procedures

Female Yorkshire/Yorkshire cross pigs (weight range 15-40 kg) were acquired and transported to the University of Florida (UF) animal care services (ACS) facility. The animal was observed on the day/the day prior to the surgery and have formal neurological/behavioral scoring done using the neurological assessment panel consisting of standardized and validated scores (neurological alertness score (NAS), neurological dysfunction score (NDS) and overall performance score (OPC)). The pig was fasted overnight, prior to surgery.

At the beginning of the procedure, the animal was sedated with ketamine at a dose of 1-2 mg/kg intramuscularly (IM), and intravenous (IV) access was established via the ear vein. Alternatively, if ketamine alone failed to obtain good sedation, a mixture of butorphanol, azaperone and medetomidine (BAMTM) was administered at 1 mL per 100 lbs. Standard monitoring equipment was prepared/applied including frontal plane electrocardiogram (EKG), pulse oximetry (SpO2), end-tidal carbon dioxide (etCO2), temperature probe, and non-invasive blood pressure monitoring (NIBP). Anesthesia was then induced using propofol at a dose of 2-4 mg/kg and ketamine at 0.5-2 mg/kg/min. The pig was intubated with a cuffed tube and mechanically ventilated with an FiO2 of 100%. The respiratory rate was adjusted to maintain etCO2 of 35-40 mm Hg [=standard ventilation]. Anesthesia was maintained as total intravenous anesthesia (TIVA) using propofol at a dose of 10-80 mg/kg/hr and ketamine at a dose of up to 2 mg/kg/min. The pig was then placed in dorsal recumbency. Ophthalmic ointment (Puralube) was administered to prevent corneal dryness and injury at the time of anesthetic induction.

A baseline blood sample for the biomarkers ubiquitin carboxyl-terminal hydrolase L1 (UCH-L1, neuronal cell body marker), glial fibrillary acidic protein (GFAP, astroglial injury/gliosis marker), neurofilament light chain (NF-L, axonal injury marker) and total Tau/p-Tau (neurodegenerative marker) was collected [=standard biomarker panel]. Vascular access to the right and left femoral vessels and right jugular vein was secured via surgical cutdown performed using aseptic technique.

To induce ventricular fibrillation (VF) and thus cardiac arrest, a 22 G, 3.5 in spinal needle (stylet removed) connected to a 5 mL syringe was inserted perpendicular to the skin into the thoracic cavity along the left sternal border at the level of the second intercostal space. The needle was inserted using constant aspiration to detect inadvertent vascular entry to a depth of about 2-3 cm, avoiding entry into the heart or other underlying organ. Once placed correctly, the needle pulsed in time with the heartbeat. A second needle was inserted in a similar manner, immediately caudal to the xyphoid process. Then, a small electrical current (12V) was applied across the needles using alligator clips in a rapid pulsing fashion. This resulted in VF within 10 sec.

Once VF was induced, ventilation was stopped. After loss of the arterial blood pressure wave form, a no flow period of 3-5 min was observed, based on institutional experience with previous swine models of cardiac arrest and based on national EMS response time averages for cardiac arrest calls, to reflect real life human care as much as possible. Manual chest compressions (CPR) were started. The animal was ventilated with 100% FiO2 at approximately 10 breaths per min.

After 2 min of CPR, NANO2 DDFPe (NuvOx Pharma, LLC, Tucson, Arizona) was administered over 10 minutes at 0.5 mL/kg of subject body weight (for example, ˜−20 mL for a ˜40 kg swine) via the central venous catheter (CVC) in the intervention animals, whereas normal saline placebo was administered to control animals. NANO2 DDFPe was supplied by NuvOx Pharma as a liquid comprising 2% weight/volume dodecafluoropentane (DDFP) with 0.3% weight/volume PEG-telomer-B (PTB) in a saline solution with 30% weight/volume sucrose with phosphate buffered saline at near neutral pH. The animal was resuscitated using standard advanced cardiac life support (ACLS) interventions, including defibrillation with 200 J, amiodarone at a dose of 150 mg for up to two doses, epinephrine every 4 min at a dose of 1 mg. Sodium bicarbonate 8.4% at a dose of 50 mEq was given if no return of spontaneous circulation (ROSC) had been achieved after 15 min of CPR. CPR was terminated if ROSC had not been achieved after 45 min of CPR. If ROSC was achieved, standard ventilation was resumed. Hypotension was treated with intravenous infusion of norepinephrine as needed for up to two hours. If the animal did achieve stable blood pressure (mean arterial pressure (MAP) of at least 50 mm Hg) during this time period without pharmacological support, it was euthanized as outlined in the euthanasia procedure below.

Continuous measurements were obtained of EKG rhythm, SpO2, ScVO2, arterial blood pressure (ABP) throughout the preparation, arrest and post-ROSC recovery phases. Arterial blood gas (ABG) samples were obtained after intubation, before stop of ventilation, immediately before end of no-flow period, every 5 min during CPR, upon ROSC, 15 min after ROSC.

Blood samples were obtained for another standard biomarker panel as defined above after 15 min of stable ROSC (defined as no re-arrest, no runs of ventricular tachycardia, and no vasopressors support above a dose of 0.8 mcg/kg/min). An echocardiogram was obtained with a portable ultrasound machine by a trained ultrasonographer upon stable ROSC and immediately before the end of anesthesia. Note was made of ejection fraction and presence of regional wall motion abnormalities.

Anesthesia was maintained for two hours post-ROSC in surviving animals. At the end of anesthesia, prior to awakening, all catheters except for a percutaneous right cranial vena cava catheter were removed, and the animal received 0.03 mg/kg IV of buprenorphine for prevention and treatment of pain. Buprenorphine sustained release (SR) was administered for the treatment of pain at induction at a dose of 0.12-0.24 mg/kg SQ every 72 hrs as needed. Breakthrough pain if present, could be treated with standard buprenorphine at 0.01-0.05 mg/kg IM every 8-12 hours pro re nata for the remainder of the study period.

Based on previous experiences with swine cardiac arrest models at the UF ACS, the animal was placed into a padded cage upon termination of anesthesia to prevent self-injury during the awakening and post-experiment recovery phase for up to four hours or overnight depending on the neurologic status of the pig (as recommended by the veterinary staff). The animal was assessed every 15 min while in the padded cage during initial recovery. Afterwards, the animal was returned to the housing area. There, the animal was observed the animal up to every 12 hours to document its neurological status. Neurological function was assessed via standardized and validated scores (neurological alertness score (NAS), neurological dysfunction score (NDS) and overall performance score (OPC)). Additional biomarker panels were collected at 24, 48, 72 and 96 hours via the IV in place.

At 96 hours, the animal was euthanized using sodium pentobarbital with phenytoin (Euthasol) at a dose of 150 mg/kg IV. After death was confirmed, the animal's brain was collected for further pathology/histology studies. Specifically, brain tissue was sent to UF Pathology for preparation in 4% formaldehyde as 3-5 mm coronal slices of the hippocampal CA1 sector and cortex, as areas that are particularly prone to hypoxic and anoxic insults. Tissues were stained with hematoxylin and eosin. Light microscopy was used to assess for severity and extent of ischemic neuronal change, infarcts, and edema. Findings were documented in a standardized fashion by using the histologic damage score described by Janata, 2010, supra. This score has previously been shown to correlate with NAS and NDS scores.

Crude ROSC and survival rates were as follows:
Placebo (n=5): ROSC 2/4 (40%), survival 1/5 (20%)
Verum (NANO2 DDFPe, n=7): 6/7 (85%), survival 4/7 (57%)

### Example 2: Use of Oxygent PFC in an Adult Swine Model of Cardiac Arrest

Oxygent is a perfluorochemical-based oxygen carrier [68, 69]. Oxygent PFC was tested in an adult swine model of cardiac arrest.

The anesthetized pig was placed in dorsal recumbency on the operating room table. The animal was monitored throughout the case on the cardiac monitor. TIVA was provided with continuous infusions of ketamine and propofol. The right neck area was prepped and an intravascular catheter was placed. A baseline 10 mL biomarker blood sample was then collected. Attention was then directed to the inguinal area. Donned sterile gown, sterile gloves, face shield, bouffant, shoe covers. The area was cleaned ×3 with chlorhexidine. A dual lumen 7Fr central venous catheter was placed under ultrasonographic real-time guidance on first attempt into the femoral vein. Attention was then directed to the chest area. An echocardiogram revealed mildly depression LVEF (left ventricular ejection fraction), normal RV (right ventricle) function, nor regional wall motion abnormalities (RWMA). The chest area was cleaned ×3 with chlorhexidine. A spinal needle was advanced 1.5 cm into the thoracic cavity in the parasternal second intercostal space. A second spinal was then advanced 1.5 cm into the thoracic cavity in the immediate subxiphoid area. Three 9V batteries were connected to the spinal needles via wires and alligator clips, resulting in ventricular fibrillation at 1119 hours. Ventilation was stopped and a no-flow phase of 3 min was observed. CPR was then initiated with good waveform and blood pressure noted on invasive monitoring. Manual bagging was initiated along with CPR as per the protocol. After 2 min of CPR, the animal was defibrillated with 50 J, followed by another 2 min of CPR. At this time, return of spontaneous circulation (ROSC) was noted. Mechanical ventilation was resumed. Perfluorocarbon was injected immediately after the first two min of CPR (Oxygent PFC, (Alliance Pharmaceuticals, San Diego, CA), oxygenated, 100 mL, followed by normal saline flush, via the femoral central venous catheter). At 15 min after ROSC, another 10 ml blood sample for biomarkers was taken. Blood samples were taken for ABGs (arterial blood gases), due to cartridge malfunction, only one ABG (arterial blood gas) resulted about 20 min after ROSC: pH 7.085, PCO2 (partial pressure of carbon dioxide) 70.4, P02 (partial pressure of oxygen) 263, BE (base excess) −9, HCO3 (bicarbonate) 21.1, TCO2 (total carbon dioxide) 23, S02 (oxygen saturation) 100%, Na (sodium) 139, K 4.4 (potassium), iCa (ionized calcium) 1.26, Hct (hematocrit) 37, Hgb (hemoglobin) 12.6. The ventilator was adjusted and NaHCO₃ (sodium bicarbonate) was administered IV. Serial ultrasound assessment demonstrated normal to hyperdynamic LVEF, normal RV function, no definitive RWMA, lung sliding with A profile. Lidocaine and amiodarone were used for profound tachycardia. Norepinephrine was initiated for hypotension and was weaned off towards the end of the 2-hour anesthesia period. As anesthesia was weaned, it was attempted to place a percutaneous cava/jugular vein line under ultrasound, but this was aborted. The animal was transported from the OR (operating room) to padded cage by ACS staff where supplemental oxygen was provided via the ETT (endotracheal u) Hyperthermia was noted and treated with external ice packs and midazolam. The animal did not recover to the point of sufficient spontaneous ventilation and therefore the decision was made to proceed with euthanasia; Euthasol was administered at 1454 hrs.

### Example 3: Use of Dodecafluoropentane Emulsion in Human with Cardiac Arrest

NANO2 DDFPe is used in the resuscitations algorithms for patients suffering from cardiac arrest, in addition to currently used standard ACLS medications. NANO2 DDFPe is administered within about 10 to about 30 minutes of cardiac arrest, for example by hospital staff in case of in-hospital cardiac arrest or by paramedics in out-of-hospital cardiac arrest, once vascular access has been established by hospital staff or paramedics on-scene, during the first minutes of resuscitation.

### Example 4: Methods

### Summary

Twelve female Yorkshire/Yorkshire cross pigs were included for analysis. Two 22Ga spinal needles were inserted along the left sternal border and caudal to the left xiphoid process. Rapid 12V shocks induced ventricular fibrillation and CPR was initiated after 5-minute downtime. DDFPe was administered via slow hand push during resuscitation. Serum biomarkers for neurologic injury were drawn before induction, after ROSC and every 24 hours in surviving animals. Formal neurological-behavioral scoring using the Neurological Alertness Score, Neurological Dysfunction Score, and Overall Performance Score was performed. Histopathology for cellular injury was performed.

### Study Population

This study was approved by the University of Florida Animal Care and Use Committee as protocol #201810157. Twelve prepubertal female Yorkshire/Yorkshire cross pigs (weight 35-40 kg) were included. The first two animals received DDFPe in an unblinded fashion as part of initial proof of concept, and all subsequent animals were randomized to either DDFPe (n=2+5=7) vs. placebo (n=5). Animals were acclimated and observed for illness for 7 days prior to the surgery per local protocols.

### Animal Preparation and Experimental Protocol

Indomethacin was administered for 48 hours prior to surgery for all animals to prevent the species-specific pulmonary inflammatory response to emulsion particulates.[7] Animals were observed the day prior to the experiment for formal neurological-behavioral scoring using standardized and validated scores, including the Neurological Alertness Score (NAS), Neurological Dysfunction Score (NDS), and Overall Performance Score (OPC).[29-34]

Animals were sedated with ketamine at a dose of 1-2 mg/kg IM, and IV access was established in an ear vein. If ketamine failed to obtain good sedation, a mixture of butorphanol, azaperone, and medetomidine was administered at 1 mL per 45 kg. Standard monitoring equipment including frontal plane electrocardiogram (ECG), pulse oximetry (SpO2), end-tidal carbon dioxide (etCO2), temperature probe, and non-invasive blood pressure monitoring (NIBP) were applied. Anesthesia was induced and maintained using intravenous propofol and ketamine. Ophthalmic ointment was administered to prevent corneal dryness and injury. The pig was intubated with a 7.0 mm cuffed endotracheal tube and mechanically ventilated with an FiO2 of 100%. Respiratory rate was adjusted to maintain etCO2 of 35-40 mmHg. Pigs were then placed in dorsal recumbency within a cradle and the Thumper® compression device (Michigan Instruments, Kentwood, MI, USA).

Baseline blood biomarker panel was then collected including ubiquitin C-terminal hydrolase L1 (UCH-L1, neuronal cell body marker), glial fibrillary acidic protein (GFAP, astroglial injury, and gliosis marker), neurofilament light chain (NFL (NF-L), axonal injury marker) and Tau protein (neurodegenerative marker).[35,36] Vascular access via ultrasound-guided Seldinger technique to the right and left femoral arteries for blood sampling and continuous blood pressure monitoring and right jugular vein for central venous access was obtained.[37] Baseline echocardiogram for ejection fraction and presence of regional wall motion abnormalities were acquired utilizing GE VScan Dual™ General Electric, Boston, MA, USA) and Butterfly iQ+™ (Butterfly Network, Burlington, MA, USA) handheld ultrasound machines.

Ventricular fibrillation (VF) and cardiac arrest was induced through a method adapted from Babini et al.[38] Two 22 Ga 8.9 cm spinal needles connected to 5 mL syringes were inserted to a depth of ˜3 cm perpendicular to the skin into the thoracic cavity along the left sternal border at the level of the third intercostal space and caudal to the left xiphoid process. Constant aspiration was applied to detect inadvertent vascular or lung injury and avoid entry into the heart. Once synchronous pulsation with the heartbeat was assured, electrical current (12V) was applied across the needles using alligator clips in a rapid pulsing fashion until VF induced on ECG and decreased arterial blood pressure detected.

Once VF was induced, ventilation was stopped. Upon loss of the arterial blood pressure waveform, a no-flow period of 5 minutes was observed. This time was based on institutional experience with previous swine models of cardiac arrest and national EMS response time averages for cardiac arrest to emulate real-world care. At the 5-minute mark, chest compressions were begun using the Thumper® device and resumed ventilation with 100% FiO2 at 10 breaths per minute.

After 2 minutes of CPR, 10 ml of 4° C. DDFPe (NuvOx Pharma LLC, Tucson, AZ, USA) was administered by slow-hand push (over 5 minutes) via the jugular central line in the intervention animals, whereas 10 ml of 4° C. normal saline placebo was administered to controls. Study drug syringes were covered with opaque tape to blind the team present for the experiment and subsequent neurologic scoring to which formulation was given. The animal was resuscitated using standard advanced cardiac life support (ACLS) interventions including defibrillation with 200 J, amiodarone at a dose of 150 mg for up to two doses, and epinephrine every 4 min at doses of 1 mg. Sodium bicarbonate 8.4% at a dose of 50 mEq was given if no return of spontaneous circulation (ROSC) had been achieved after 15 min of CPR. CPR was terminated if ROSC had not been achieved after 45 min of CPR. If ROSC was achieved, ventilation to etCO2 of 35-40 mmHg was targeted. Crystalloids and infusion of norepinephrine were used for up to two hours to maintain mean arterial pressure (MAP) of at least 50 mmHg. If the animal did not achieve goal MAP by 2 hours, euthanasia was proceeded with.

Continuous ECG rhythm, SpO2, and arterial blood pressure (ABP) measurements were obtained throughout the preparation, arrest, and post-ROSC recovery phases. Arterial blood gas (ABG) samples were obtained using the i-STAT 1@blood gas analyzer (Abbott Laboratories, Abbott Park, IL, USA). Timepoints included intubation, before the stop of ventilation, at the end of the no-flow period, every 5 min during CPR, upon ROSC, and 15 min after stable ROSC (defined as no re-arrest, no ventricular tachycardia, and norepinephrine requirement below 0.8 meg/kg/min).

Repeat echocardiogram upon stable ROSC was performed, evaluating ejection fraction, regional wall motion abnormalities, and presence of post-CPR pneumothorax.

Anesthesia was maintained for two hours post-ROSC in surviving animals. At the end of anesthesia, prior to awakening, all catheters except for a percutaneous right jugular catheter were removed, and animals received IV buprenorphine for analgesia. Buprenorphine sustained release was administered for the treatment of pain every 72 hours as needed. Breakthrough pain, if present, was treated with standard pro re nata for the remainder of the study period.

Animals were placed into a padded cage upon termination of anesthesia for four hours or overnight, depending on neurologic status and attending veterinarian discretion, to prevent self-injury during the post-experiment recovery phase. Animals were assessed every 15 min while in the padded cage during initial recovery. Afterward, animals were returned to the housing area. Animals were observed at regular intervals to document neurological status. Blood samples for biomarker panels were obtained at 24-hour intervals.

At 96 hours, animals were euthanized using sodium pentobarbital with phenytoin. After death was confirmed, brains were collected for further pathology/histology studies from the animals that had received DDFPe.

### Functional Assessment

Surviving animals underwent serial exams for 96 hours using the neurological assessment panel described previously. Exams were performed every twelve hours by an examiner trained in the use of our neurological assessment panel and blinded to study arm assignment. Upon regaining completely intact neurological scores for two consecutive assessments, exam intervals were extended to every 24 hours.

### Histopathology

Upon euthanasia, the brain tissue was assessed for the degree of cell injury. Brain tissue samples included parietal cortex and hippocampus from both hemispheres and were removed and fixed in neutral buffered formalin for 7 days.[36] After fixation, the tissues were rosined and stored in phosphate-buffered saline. Tissues were embedded in paraffin, and coronal sections (5 μm) were cut for hematoxylin and eosin staining as well as for GFAP immunohistochemistry staining in the core facility of Molecular Pathological Center, University of Florida.[37] Digital images were obtained by a Nikon Eclipse (E600) microscope with NIS-Elements software (Nikon Instruments, Melville, NY, USA). The neurons and astrocytes in the areas of the hippocampal CA1 region and cortex are particularly prone to hypoxic and anoxic insults. The team's neuroanatomist used light microscopy to assess severity and extent of ischemic neuronal change, infarcts, and edema. Findings were documented in a standardized fashion by using a histologic damage score described in the literature.[33] This score has previously been shown to correlate with NAS and NDS scores in pigs.[29]

### Biomarker Assays

GFAP, UCHL1, Tau, NF-L Assays (Quanterix SiMoA ELISA)

GFAP, UCHL1, Tau, NF-L (Quanterix SiMoA) were assayed with an ultrasensitive immunoassay using digital array technology (Single Molecule Arrays, SiMoA)-based Human Neurology 4-Plex B assay (N4PB; Item 103345). pNF-H Assay was run on the SiMoA discovery kit (Item 102669). All SiMoA assays were run on the SR-X benchtop assay platform (Quanterix Corp., Lexington, MA) at the University of Florida (Gainesville, Florida) and Morehouse School of Medicine (Neurobiology, Center for Neurotrauma, Multiomics & Biomarkers) according to manufacturer instructions. The LLOQ, LOD and dynamic range are 9.38 pg/mL, 1.32 pg/mL and 1.32 to 250,000 μg/mL for both GFAP and UCH-L, respectively. Interassay and intraassay % CV are 7.5-10.8% and 6.8-10.5%, respectively for GFAP. Interassay and intraassay % CV are 3.5-8.8% and 6.3-11.7%, respectively for UCH-L1. The LLOQ, LOD and dynamic range are. 0.625 pg/mL, 0.097 pg/mL and 0.0971-10,000 μg/mL for NF-L, respectively. Interassay and intraassay % CV are 4.6-6.9% and 3.5-7.5%, respectively for NF-L. The LLOQ, LOD and dynamic range are. 0.0236 pg/mL, 0.05 pg/mL and 0.0971-400 pg/mL for Tau (Quanterix), respectively. Interassay and intraassay % CV are 4.8-10.0% and 2.1-8.8%, respectively for Tau (Quanterix) respectively.

### Statistical Analysis

Data visualization with line plots was used for the initial analysis of biomarker and blood gas data. For each biomarker, two plots were made: the first showing the trend over the full observation period, and the second focusing on the first 2 hours after arrest. Functional neurologic scoring was visualized using heatmaps per animal.

Differences in ROSC and survival rates were tested with Fisher's exact test. To assess whether changes in biomarker levels over time differed between placebo and verum groups, linear mixed-effects models with subject-specific random intercepts were used to test the interactions between treatment and time. All analyses were performed using R Statistical Software version 4.1.2[39]. Linear mixed-effects models were fitted using the lme4 R package; plots were drawn with the ggplot2 R package [40, 41].

### Example 5

### Results

Results Summary Five of twelve animals survived to the 96-hour observation period including one placebo and four DDFPe. For ROSC, the odds ratio between DDFPe and placebo group was 7 218 (p-value 0 222) For survival, the odds ratio between DDFPe and placebo group was 4.609 (p-value 0.293). Surviving animals all had decreased motor responses that recovered to sustained normality by 72 hours post-ROSC. DDFPe animals showed less neuronal damage than controls. Infarction was not found in any brains on autopsy or on H&E staining.

### Resuscitation

Baseline hemodynamic and biomarker data between the placebo and DDFPe groups were similar, and all animals scored full points for functional neurologic assessment before the procedure. Of 12 experiments, 7 animals were given DDFPe, and 5 animals received a placebo. Out of 5 placebo animals, 2 (40%) achieved ROSC, and 1 (20%) survived to 96 hours. Out of 7 DDFPe animals, 6 (85%) achieved ROSC, and 4 (57%) survived to 96 hours. The odds ratio of ROSC comparing the DDFPe and placebo groups was 7.2 18 (p-value 0.222). The odds ratio of survival comparing the DDFPe and placebo groups was 4,609 (p-value 0.293). There was a significant difference in the duration of CPR and dosage of epinephrine, bicarbonate, and defibrillation attempts between groups with DDFPe animals achieving stable ROSC sooner, avoiding the need for additional epinephrine, bicarbonate, or prolonged resuscitation.

Arterial blood gas sampling showed significant heterogeneity of treatment effects between animals and groups, so formal statistical testing was not performed. Tables 1-4 present blood gas measurements for each animal at times indicated (legend for times in Table 5).

Abbreviations in Tables 1-4: Na (sodium); K (potassium); Cl (chloride); iCa (ionized calcium); TCO2 (total carbon dioxide) Gluc (glucose); BUN (blood urea nitrogen); Creat (creatinine); Hct (hematocrit); Hb (hemoglobin); AnGap (anion gap); Temp (temperature in ° Celsius); pH; PCO2 (partial pressure of carbon dioxide); P02 (partial pressure of oxygen); BEecf (base excess in the extracellular fluid compartnent); HCO3 (bicarbonate); sO2 (oxygen saturation); Lac (lactate).

Table 1: Column 1 (Subject); Column 2 (Treatment, DDFPe-treated (verum), placebo-treated (placebo)); Column 3 (Time); Column 4 (Na (mmol/L)); Column 5 (K (mmol/L)); Column 6 (Cl (mmol/L)); Column 7 (iCa (mmol/L)); Column 8 (TCO2 (mmol/L))

Table 2: Column 1 (Subject); Column 2 (Treatment, DDFPe-treated (verum), placebo-treated (placebo)); Column 3 (Time); Column 4 (Glucose (mg/dL)); Column 5 (BUN (mg/dL)); Column 6 (Creat (mg/dL)); Column 7 (Hematocrit (%)); Column 8 (Hemoglobin (g/dL))

Table 3: Column 1 (Subject); Column 2 (Treatment, DDFPe-treated (verum), placebo-treated (placebo)); Column 3 (Time); Column 4 (Anion Gap (mmol/L)); Column 5 (Temp (Celsius)); Column 6 (pH); Column 7 (PCO2 (mmHg)); Column 8 (P02 (mmHg))

Table 4: Column 1 (Subject); Column 2 (Treatment, DDFPe-treated (verum), placebo-treated (placebo)); Column 3 (Time); Column 4 (Base Excess (mmol/L)); Column 5 (HCO3 (mmol/L)); Column 6 (saturation 02(%)); Column 7 (Lactate (mmol/L)); Column 8 (TCO2 (mmol/L))

TABLE 1
Blood Gas Measurement: Na, K, Cl, ICa, and TCO2

| Subject | | | Na | K | Cl | iCa | TCO2 |
|---|---|---|---|---|---|---|---|
| Number | Treatment | Time | (mmol/L) | (mmol/L) | (mmol/L) | (mmol/L) | (mmol/L) |
| 1 | Verum | Baseline | 147 | 2.4 | 113 | 1.06 | 24 |
| 1 | Verum | PreVfib | | | | | |
| 1 | Verum | NoFlow | | | | | |
| 1 | Verum | ROSC | | | | | 28 |
| 2 | Verum | Baseline | 139 | 4 | 101 | 1.35 | 31 |
| 2 | Verum | PreVfib | | | | | |
| 2 | Verum | NoFlow | | | | | |
| 2 | Verum | CPR5 | | | | | 26 |
| 2 | Verum | ROSC | | | | | 19 |
| 2 | Verum | ROSC15 | 139 | 4.2 | 105 | 1.22 | 19 |
| 2 | Verum | ROSC30 | | | | | |
| 2 | Verum | ROSC45 | | | | | |
| 2 | Verum | ROSC60 | | | | | |
| 2 | Verum | ROSC75 | | | | | 26 |
| 2 | Verum | PreEuth | 142 | 4.4 | | 1.14 | 34 |
| 3 | Placebo | Baseline | 137 | 4.2 | 97 | 1.46 | |
| 3 | Placebo | PreVfib | 137 | 4 | 98 | 1.41 | |
| 3 | Placebo | NoFlow | 137 | 4 | 97 | 1.43 | |
| 3 | Placebo | CPR5 | 134 | 7.2 | 101 | 1.42 | |
| 3 | Placebo | CPR10 | 131 | 8.5 | 95 | 0.96 | |
| 4 | Verum | Baseline | 141 | 3.8 | | 1.39 | 37 |
| 4 | Verum | PreVfib | 140 | 3.8 | | 1.43 | 36 |
| 4 | Verum | NoFlow | | | | | |
| 4 | Verum | CPR5 | 138 | 4.7 | | 1.37 | 33 |
| 4 | Verum | CPR10 | 135 | 7.8 | | 1.35 | 28 |
| 4 | Verum | ROSC | 139 | 6 | | 1.4 | 31 |
| 4 | Verum | ROSC15 | 142 | 3 | | 1.31 | 28 |
| 4 | Verum | ROSC30 | 141 | 3.7 | | 1.3 | 30 |
| 5 | Verum | Baseline | 140 | 3.7 | | 1.39 | 41 |
| 5 | Verum | PreVfib | 140 | 3.8 | | 1.4 | 39 |
| 5 | Verum | NoFlow | | | | | |
| 5 | Verum | CPR5 | 136 | 6.8 | | 1.33 | 29 |
| 5 | Verum | CPR10 | 137 | 7.6 | | 1.37 | 29 |
| 5 | Verum | ROSC | 137 | 6.9 | | 1.37 | 28 |
| 5 | Verum | ROSC15 | 144 | 4.6 | | 1.21 | 24 |
| 5 | Verum | ROSC30 | 148 | 3.5 | | 1.2 | 28 |
| 5 | Verum | PreEuth | 147 | 4.1 | | 1.26 | 29 |
| 6 | Placebo | Baseline | 142 | 3.8 | | 1.53 | 33 |
| 6 | Placebo | PreVfib | 141 | 3.9 | | 1.52 | 33 |
| 6 | Placebo | NoFlow | 139 | 5.6 | | 1.47 | 31 |
| 6 | Placebo | CPR5 | 138 | 7 | | 1.43 | 30 |
| 6 | Placebo | CPR10 | 138 | 7.1 | | 1.52 | 25 |
| 6 | Placebo | CPR15 | 152 | 2.4 | | 0.73 | 8 |
| 6 | Placebo | CPR20 | 151 | 4 | | 1.01 | 20 |
| 6 | Placebo | CPR25 | 157 | 1.9 | | 0.38 | 4 |
| 6 | Placebo | CPR30 | 143 | 7.7 | | 1.14 | 21 |
| 6 | Placebo | CPR35 | 150 | 5.8 | | 0.82 | 13 |
| 6 | Placebo | CPR40 | 147 | 7.4 | | 0.87 | 15 |
| 7 | Placebo | Baseline | 141 | 3.6 | | 137 | 3.5 |
| 7 | Placebo | PreVfib | 140 | 3.6 | | 1.38 | 36 |
| 7 | Placebo | NoFlow | | | | | |
| 7 | Placebo | CPR5 | 139 | 5.2 | | 1.3 | 28 |
| 7 | Placebo | ROSC | 139 | 5 | | 1.39 | 26 |
| 7 | Placebo | ROSC15 | 141 | 3.1 | | 1.25 | 25 |
| 7 | Placebo | PreEuth | 143 | 4 | | 1.26 | 28 |
| 8 | Verum | Baseline | 141 | 3.8 | | 1.4 | 34 |
| 8 | Verum | PreVfib | 140 | 3.8 | | 1.43 | 36 |
| 8 | Verum | NoFlow | | | | | |
| 8 | Verum | CPR5 | 140 | 4.3 | | 1.41 | 34 |
| 8 | Verum | CPR10 | 137 | 6 | | 1.25 | 25 |
| 8 | Verum | CPR15 | 135 | 6.8 | | 1.18 | 21 |
| 8 | Verum | CPR20 | 133 | 7.4 | | 1.2 | 20 |
| 8 | Verum | CPR25 | 151 | 7.7 | | 0.95 | 51 |
| 8 | Verum | CPR30 | 138 | 9.1 | | 1.04 | 32 |
| 8 | Verum | CPR35 | 136 | 9.1 | | 1.05 | 27 |
| 8 | Verum | CPR40 | 135 | 9.1 | | 1.02 | 23 |
| 9 | Placebo | Baseline | 138 | 3.8 | | 1.43 | 39 |
| 9 | Placebo | PreVfib | | | | | |
| 9 | Placebo | NoFlow | 139 | 3.9 | | 1.44 | 35 |
| 9 | Placebo | CPR5 | 133 | 7.7 | | 1.41 | 27 |
| 9 | Placebo | CPR10 | 137 | 6.6 | | 1.26 | 21 |
| 9 | Placebo | CPR15 | 149 | 3.9 | | 0.86 | 12 |
| 9 | Placebo | CPR20 | 151 | 5.4 | | 0.94 | 25 |
| 9 | Placebo | CPR25 | | | | | |
| 9 | Placebo | CPR30 | 162 | 2.5 | | 0.46 | 12 |
| 9 | Placebo | CPR35 | | | | | |
| 9 | Placebo | CPR40 | | | | | |
| 9 | Placebo | CPR45 | 145 | 8.4 | | 0.99 | 27 |
| 10 | Placebo | Baseline | 137 | 4.3 | | 1.26 | 35 |
| 10 | Placebo | PreVfib | 138 | 4.3 | | 1.29 | 37 |
| 10 | Placebo | NoFlow | 135 | 6.9 | | 1.28 | 30 |
| 10 | Placebo | ROSC | 136 | 6.5 | | 1.27 | 28 |
| 10 | Placebo | ROSC15 | 139 | 3.3 | | 1.23 | 27 |
| 10 | Placebo | ROSC30 | 139 | 3.7 | | 1.15 | 28 |
| 10 | Placebo | ROSC45 | 138 | 3.9 | | 1.12 | 29 |
| 11 | Verum | Baseline | 142 | 4.5 | | 1.48 | 37 |
| 11 | Verum | PreVfib | | | | | |
| 11 | Verum | NoFlow | | | | | |
| 11 | Verum | ROSC | 141 | 4.3 | | 1.43 | |
| 11 | Verum | ROSC15 | 144 | 3.1 | | 1.42 | 27 |
| 11 | Verum | ROSC30 | 144 | 3.3 | | 1.39 | 25 |
| 11 | Verum | ROSC45 | 143 | 3.9 | | 1.4 | 26 |
| 11 | Verum | ROSC60 | 142 | 4.5 | | 1.39 | 28 |
| 11 | Verum | ROSC75 | 143 | 4.8 | | 1.46 | 33 |
| 12 | Verum | Baseline | 136 | 4.9 | | 1.31 | 36 |
| 12 | Verum | PreVfib | 137 | 4.5 | | 1.37 | 33 |
| 12 | Verum | NoFlow | 137 | 4.7 | | 1.37 | 30 |
| 12 | Verum | CPR5 | 136 | 6.3 | | 1.25 | 26 |
| 12 | Verum | ROSC | 136 | 6.4 | | 1.26 | 22 |
| 12 | Verum | ROSC15 | 139 | 3.9 | | 1.29 | 25 |
| 12 | Verum | ROSC30 | 138 | 4.2 | | 1.3 | 26 |
| 12 | Verum | ROSC45 | 139 | 4.1 | | 1.22 | 25 |

TABLE 2
Blood Gas Measurement: Glucose, BUN, Create, Hematocrit, Hemoglobin

| Subject | | | Glucose | BUN | Creat | Hematocrit | Hemoglobin |
|---|---|---|---|---|---|---|---|
| Number | Treatment | Time | (mg/dL) | (mg/dL) | (mg/dL) | (%) | (g/dL) |
| 1 | Verum | Baseline | 53 | 2.9 | 0.5 | 14 | |
| 1 | Verum | PreVfib | | | | | |
| 1 | Verum | NoFlow | | | | | |
| 1 | Verum | ROSC | | | | | |
| 2 | Verum | Baseline | 115 | 5 | 1 | 26 | 8.8 |
| 2 | Verum | PreVfib | | | | | |
| 2 | Verum | NoFlow | | | | | |
| 2 | Verum | CPR5 | | | | | |
| 2 | Verum | ROSC | | | | | |
| 2 | Verum | ROSC15 | 150 | 6 | 1.2 | 28 | 9.5 |
| 2 | Verum | ROSC30 | | | | | |
| 2 | Verum | ROSC45 | | | | | |
| 2 | Verum | ROSC60 | | | | | |
| 2 | Verum | ROSC75 | | | | | |
| 2 | Verum | PreEuth | | | | 22 | 7.5 |
| 3 | Placebo | Baseline | 97 | 6 | 1.4 | 23 | 7.8 |
| 3 | Placebo | PreVfib | 99 | 6 | 1.4 | 23 | 7.8 |
| 3 | Placebo | NoFlow | 98 | 6 | 1.3 | 22 | 7.5 |
| 3 | Placebo | CPR5 | 253 | 7 | 1.4 | 25 | 8.5 |
| 3 | Placebo | CPR10 | 245 | 9 | 1.5 | 14 | |
| 4 | Verum | Baseline | 74 | | | 23 | 7.8 |
| 4 | Verum | PreVfib | 72 | | | 23 | 7.8 |
| 4 | Verum | NoFlow | | | | | |
| 4 | Verum | CPR5 | 68 | | | 22 | 7.5 |
| 4 | Verum | CPR10 | 67 | | | 30 | 10.2 |
| 4 | Verum | ROSC | 64 | | | 33 | 11.2 |
| 4 | Verum | ROSC15 | 66 | | | 30 | 10.2 |
| 4 | Verum | ROSC30 | 70 | | | 27 | 9.2 |
| 5 | Verum | Baseline | 82 | | | 23 | 7.8 |
| 5 | Verum | PreVfib | 81 | | | 22 | 7.5 |
| 5 | Verum | NoFlow | | | | | |
| 5 | Verum | CPR5 | 108 | | | 28 | 9.5 |
| 5 | Verum | CPR10 | 131 | | | 30 | 10.2 |
| 5 | Verum | ROSC | 108 | | | 28 | 9.5 |
| 5 | Verum | ROSC15 | 61 | | | 20 | 6.8 |
| 5 | Verum | ROSC30 | 48 | | | 19 | 6.5 |
| 5 | Verum | PreEuth | 44 | | | 19 | 6.5 |
| 6 | Placebo | Baseline | 99 | | | 26 | 8.8 |
| 6 | Placebo | PreVfib | 99 | | | 26 | 8.8 |
| 6 | Placebo | NoFlow | 105 | | | 27 | 9.2 |
| 6 | Placebo | CPR5 | 219 | | | 33 | 11.2 |
| 6 | Placebo | CPR10 | 301 | | | 35 | 11.9 |
| 6 | Placebo | CPR15 | 91 | | | 14 | |
| 6 | Placebo | CPR20 | 179 | | | 21 | 7.1 |
| 6 | Placebo | CPR25 | 45 | | | 14 | |
| 6 | Placebo | CPR30 | 240 | | | 23 | 7.8 |
| 6 | Placebo | CPR35 | 142 | | | 14 | |
| 6 | Placebo | CPR40 | 157 | | | 14 | |
| 7 | Placebo | Baseline | 88 | | | 23 | 7.8 |
| 7 | Placebo | PreVfib | 88 | | | 23 | 7.8 |
| 7 | Placebo | NoFlow | | | | | |
| 7 | Placebo | CPR5 | 105 | | | 23 | 7.8 |
| 7 | Placebo | ROSC | 259 | | | 32 | 10.9 |
| 7 | Placebo | ROSC15 | 256 | | | 27 | 9.2 |
| 7 | Placebo | PreEuth | 107 | | | 21 | 7.1 |
| 8 | Verum | Baseline | 94 | | | 23 | 7.8 |
| 8 | Verum | PreVfib | 99 | | | 26 | 8.8 |
| 8 | Verum | NoFlow | | | | | |
| 8 | Verum | CPR5 | 89 | | | 26 | 8.8 |
| 8 | Verum | CPR10 | 98 | | | 23 | 7.8 |
| 8 | Verum | CPR15 | 131 | | | 23 | 7.8 |
| 8 | Verum | CPR20 | 162 | | | 24 | 8.2 |
| 8 | Verum | CPR25 | 169 | | | 19 | 6.5 |
| 8 | Verum | CPR30 | 187 | | | 25 | 8.5 |
| 8 | Verum | CPR35 | 165 | | | 25 | 8.5 |
| 8 | Verum | CPR40 | 123 | | | 26 | 8.8 |
| 9 | Placebo | Baseline | 90 | | | 21 | 7.1 |
| 9 | Placebo | PreVfib | | | | | |
| 9 | Placebo | NoFlow | 88 | | | 20 | 6.8 |
| 9 | Placebo | CPR5 | 193 | | | 27 | 9.2 |
| 9 | Placebo | CPR10 | 167 | | | 24 | 8.2 |
| 9 | Placebo | CPR15 | 90 | | | 14 | |
| 9 | Placebo | CPR20 | 119 | | | 18 | 6.1 |
| 9 | Placebo | CPR25 | | | | | |
| 9 | Placebo | CPR30 | 43 | | | 14 | |
| 9 | Placebo | CPR35 | | | | | |
| 9 | Placebo | CPR40 | | | | | |
| 9 | Placebo | CPR45 | 100 | | | 20 | 6.8 |
| 10 | Placebo | Baseline | 91 | | | 21 | 7.1 |
| 10 | Placebo | PreVfib | 91 | | | 21 | 7.1 |
| 10 | Placebo | NoFlow | 99 | | | 23 | 7.8 |
| 10 | Placebo | ROSC | 101 | | | 30 | 10.2 |
| 10 | Placebo | ROSC15 | 94 | | | 29 | 9.9 |
| 10 | Placebo | ROSC30 | 96 | | | 25 | 8.5 |
| 10 | Placebo | ROSC45 | 99 | | | 22 | 7.5 |
| 11 | Verum | Baseline | 189 | | | 26 | 8.8 |
| 11 | Verum | PreVfib | | | | | |
| 11 | Verum | NoFlow | | | | | |
| 11 | Verum | ROSC | 169 | | | 39 | 13.3 |
| 11 | Verum | ROSC15 | 167 | | | 33 | 11.2 |
| 11 | Verum | ROSC30 | 156 | | | 34 | 11.6 |
| 11 | Verum | ROSC45 | 167 | | | 31 | 10.5 |
| 11 | Verum | ROSC60 | 169 | | | 31 | 10.5 |
| 11 | Verum | ROSC75 | 176 | | | 31 | 10.5 |
| 12 | Verum | Baseline | 104 | | | 26 | 8.8 |
| 12 | Verum | PreVfib | 94 | | | 24 | 8.2 |
| 12 | Verum | NoFlow | 89 | | | 24 | 8.2 |
| 12 | Verum | CPR5 | 139 | | | 26 | 8.8 |
| 12 | Verum | ROSC | 193 | | | 26 | 8.8 |
| 12 | Verum | ROSC15 | 196 | | | 26 | 8.8 |
| 12 | Verum | ROSC30 | 187 | | | 26 | 8.8 |
| 12 | Verum | ROSC45 | 158 | | | 23 | 7.8 |

TABLE 3
Blood Gas Measurement: Anion Gap, Temperature, pH, PCO2, PO2

| | | | Anion | | | | |
|---|---|---|---|---|---|---|---|
| Subject | | | Gap | Temp | | PCO2 | PO2 |
| [symbol] | Treatment | Time | (mmol/L) | (Celsius) | pH | (mmHg) | (mmHg) |
| 1 | Verum | Baseline | 13 | 37 | 7.347 | 49.6 | 541 |
| 1 | Verum | PreVfib | | | | | |
| 1 | Verum | NoFlow | | | | | |
| 1 | Verum | ROSC | | 37 | 7.354 | 48.2 | 495 |
| 2 | Verum | Baseline | 12 | 37 | 7.486 | 39.9 | 413 |
| 2 | Verum | PreVfib | | | | | |
| 2 | Verum | NoFlow | | | | | |
| 2 | Verum | CPR5 | | 37 | 7.27 | 54 | 299 |
| 2 | Verum | ROSC | | 37 | 6.974 | 72.2 | 166 |
| 2 | Verum | ROSC15 | 19 | 37 | 7.102 | 55.3 | 251 |
| 2 | Verum | ROSC30 | | | | | |
| 2 | Verum | ROSC45 | | | | | |
| 2 | Verum | ROSC60 | | | | | |
| 2 | Verum | ROSC75 | | 37 | 7.249 | 55.7 | 400 |
| 2 | Verum | PreEuth | | 37 | 7.39 | 53.9 | 388 |
| 3 | Placebo | Baseline | | | 7.518 | 46.4 | 509 |
| 3 | Placebo | PreVfib | | | 7.483 | 52.4 | 554 |
| 3 | Placebo | NoFlow | | | 7.508 | 44 | 274 |
| 3 | Placebo | CPR5 | | | 7.096 | 107.6 | 42 |
| 3 | Placebo | CPR10 | | | 6.898 | 131 | |
| 4 | Verum | Baseline | | 37 | 7.426 | 54.3 | 537 |
| 4 | Verum | PreVfib | | 37 | 7.407 | 54.6 | 539 |
| 4 | Verum | NoFlow | | | | | |
| 4 | Verum | CPR5 | | 37 | 7.45 | 46 | 417 |
| 4 | Verum | CPR10 | | 37 | 7.283 | 55.8 | 112 |
| 4 | Verum | ROSC | | 37 | 7.101 | 89.9 | 89 |
| 4 | Verum | ROSC15 | | 37 | 7.26 | 57.4 | 385 |
| 4 | Verum | ROSC30 | | 37 | 7.224 | 67.1 | 464 |
| 5 | Verum | Baseline | | 37 | 7.511 | 49.6 | 519 |
| 5 | Verum | PreVfib | | 37 | 7.477 | 50.3 | 550 |
| 5 | Verum | NoFlow | | | | | |
| 5 | Verum | CPR5 | | 37 | 7.282 | 58.7 | 287 |
| 5 | Verum | CPR10 | | 37 | 7.12 | 81.5 | 61 |
| 5 | Verum | ROSC | | 37 | 7.063 | 89.2 | 71 |
| 5 | Verum | ROSC15 | | 37 | 6.933 | 99.9 | 83 |
| 5 | Verum | ROSC30 | | 37 | 6.931 | 114.8 | 84 |
| 5 | Verum | PreEuth | | 37 | 6.941 | 116.9 | 85 |
| 6 | Placebo | Baseline | | 37 | 7.426 | 48.6 | 477 |
| 6 | Placebo | PreVfib | | 37 | 7.337 | 58.7 | 592 |
| 6 | Placebo | NoFlow | | 37 | 7.303 | 59.8 | 470 |
| 6 | Placebo | CPR5 | | 37 | 7.241 | 64.9 | 172 |
| 6 | Placebo | CPR10 | | 37 | 7.061 | 80 | 74 |
| 6 | Placebo | CPR15 | | 37 | 7.137 | 21 | 74 |
| 6 | Placebo | CPR20 | | 37 | 7.24 | 44.2 | 53 |
| 6 | Placebo | CPR25 | | 37 | 7.153 | 11.3 | 44 |
| 6 | Placebo | CPR30 | | 37 | 7.035 | 71 | 45 |
| 6 | Placebo | CPR35 | | 37 | 6.914 | 55 | 57 |
| 6 | Placebo | CPR40 | | 37 | 6.846 | 72.9 | 56 |
| 7 | Placebo | Baseline | | 37 | 7.491 | 43.5 | 370 |
| 7 | Placebo | PreVfib | | 37 | 7.505 | 43.8 | 484 |
| 7 | Placebo | NoFlow | | | | | |
| 7 | Placebo | CPR5 | | 37 | 7.542 | 31.8 | 299 |
| 7 | Placebo | ROSC | | 37 | 7.134 | 70.5 | 249 |
| 7 | Placebo | ROSC15 | | 37 | 7.183 | 62.9 | 343 |
| 7 | Placebo | PreEuth | | 37 | 7.392 | 44 | 428 |
| 8 | Verum | Baseline | | 37 | 7.444 | 47.9 | 413 |
| 8 | Verum | PreVfib | | 37 | 7.434 | 52.1 | 436 |
| 8 | Verum | NoFlow | | | | | |
| 8 | Verum | CPR5 | | 37 | 7.665 | 28.9 | 269 |
| 8 | Verum | CPR10 | | 37 | 7.634 | 22.9 | 285 |
| 8 | Verum | CPR15 | | 37 | 7.429 | 29.8 | 177 |
| 8 | Verum | CPR20 | | 37 | 7.186 | 49.8 | 93 |
| 8 | Verum | CPR25 | | 37 | 7.333 | 89.6 | 45 |
| 8 | Verum | CPR30 | | 37 | 7.115 | 91.3 | 56 |
| 8 | Verum | CPR35 | | 37 | 7.049 | 87.6 | 60 |
| 8 | Verum | CPR40 | | 37 | 6.894 | 101.4 | 61 |
| 9 | Placebo | Baseline | | 37 | 7.432 | 55.3 | 475 |
| 9 | Placebo | PreVfib | | | | | |
| 9 | Placebo | NoFlow | | 37 | 7.368 | 57 | 222 |
| 9 | Placebo | CPR5 | | 37 | 7.392 | 42.9 | 155 |
| 9 | Placebo | CPR10 | | 37 | 7.271 | 43.3 | 79 |
| 9 | Placebo | CPR15 | | 37 | 7.151 | 31.2 | 53 |
| 9 | Placebo | CPR20 | | 37 | 7.268 | 52.1 | 36 |
| 9 | Placebo | CPR25 | | | | | |
| 9 | Placebo | CPR30 | | 37 | 7.303 | 21.9 | 42 |
| 9 | Placebo | CPR35 | | | | | |
| 9 | Placebo | CPR40 | | | | | |
| 9 | Placebo | CPR45 | | 37 | 6.889 | 119.9 | 25 |
| 10 | Placebo | Baseline | | 37 | 7.483 | 45.2 | 462 |
| 10 | Placebo | PreVfib | | 37 | 7.475 | 48 | 546 |
| 10 | Placebo | NoFlow | | 37 | 7.458 | 40 | 391 |
| 10 | Placebo | ROSC | | 37 | 7.33 | 49.5 | 387 |
| 10 | Placebo | ROSC15 | | 37 | 7.274 | 54.5 | 487 |
| 10 | Placebo | ROSC30 | | 37 | 7.292 | 53.8 | 510 |
| 10 | Placebo | ROSC45 | | 37 | 7.386 | 46.2 | 434 |
| 11 | Verum | Baseline | | 37 | 7.479 | 47.2 | 495 |
| 11 | Verum | PreVfib | | | | | |
| 11 | Verum | NoFlow | | | | | |
| 11 | Verum | ROSC | | 37 | | | 364 |
| 11 | Verum | ROSC15 | | 37 | 7.254 | 58.1 | 382 |
| 11 | Verum | ROSC30 | | 37 | 7.169 | 63.9 | 379 |
| 11 | Verum | ROSC45 | | 37 | 7.301 | 49.6 | 325 |
| 11 | Verum | ROSC60 | | 37 | 7.302 | 53.4 | 367 |
| 11 | Verum | ROSC75 | | 37 | 7.182 | 81.9 | 411 |
| 12 | Verum | Baseline | | 37 | 7.451 | 50 | 534 |
| 12 | Verum | PreVfib | | 37 | 7.403 | 50.7 | 511 |
| 12 | Verum | NoFlow | | | 7.382 | 47.9 | 104 |
| 12 | Verum | CPR5 | | 37 | 7.385 | 40.7 | 148 |
| 12 | Verum | ROSC | | 37 | 7.199 | 53.2 | 107 |
| 12 | Verum | ROSC15 | | 37 | 7.143 | 67.2 | 181 |
| 12 | Verum | ROSC30 | | 37 | 7.14 | 70.4 | 171 |
| 12 | Verum | ROSC45 | | 37 | 7.166 | 63.1 | 186 |

[symbol]

indicates data missing or illegible when filed

TABLE 4
Blood Gas Measurement: Base Excess, HCO3, saturation O2, Lactate

| | | | Base | | saturation | |
|---|---|---|---|---|---|---|
| Subject | | | Excess | HCO3 | O2 | Lactate |
| Number | Treatment | Time | (mmol/L) | (mmol/L) | (%) | (mmol/L) |
| 1 | Verum | Baseline | 2 | 27.2 | 100 | 0.56 |
| 1 | Verum | PreVfib | | | | |
| 1 | Verum | NoFlow | | | | |
| 1 | Verum | ROSC | 1 | 26.9 | 100 | 4.14 |
| 2 | Verum | Baseline | 7 | 30.2 | 100 | 0.44 |
| 2 | Verum | PreVfib | | | | |
| 2 | Verum | NoFlow | | | | |
| 2 | Verum | CPR5 | −2 | 24.8 | 100 | 6.62 |
| 2 | Verum | ROSC | −15 | 16.8 | 98 | 11.34 |
| 2 | Verum | ROSC15 | −12 | 17.3 | 100 | 11.32 |
| 2 | Verum | ROSC30 | | | | |
| 2 | Verum | ROSC45 | | | | |
| 2 | Verum | ROSC60 | | | | |
| 2 | Verum | ROSC75 | −3 | 24.4 | 100 | 5.21 |
| 2 | Verum | PreEuth | 8 | 32.6 | 100 | |
| 3 | Placebo | Baseline | 15 | 37.7 | 100 | 0.69 |
| 3 | Placebo | PreVfib | 16 | 39.2 | 100 | 0.51 |
| 3 | Placebo | NoFlow | 12 | 35 | 100 | 0.5 |
| 3 | Placebo | CPR5 | 3 | 33 | 55 | |
| 3 | Placebo | CPR10 | | | | 12.7 |
| 4 | Verum | Baseline | 11 | 35.7 | 100 | |
| 4 | Verum | PreVfib | 10 | 34.3 | 100 | |
| 4 | Verum | NoFlow | | | | |
| 4 | Verum | CPR5 | 8 | 32 | 100 | |
| 4 | Verum | CPR10 | 0 | 26.4 | 98 | |
| 4 | Verum | ROSC | −2 | 27.9 | 92 | |
| 4 | Verum | ROSC15 | −1 | 25.8 | 100 | |
| 4 | Verum | ROSC30 | 0 | 27.7 | 100 | |
| 5 | Verum | Baseline | 17 | 39.7 | 100 | |
| 5 | Verum | PreVfib | 14 | 37.2 | 100 | |
| 5 | Verum | NoFlow | | | | |
| 5 | Verum | CPR5 | 1 | 27.7 | 100 | |
| 5 | Verum | CPR10 | −3 | 26.5 | 80 | |
| 5 | Verum | ROSC | −5 | 25.4 | 84 | |
| 5 | Verum | ROSC15 | −11 | 21.1 | 85 | |
| 5 | Verum | ROSC30 | −8 | 24.1 | 85 | |
| 5 | Verum | PreEuth | −7 | 25.2 | 86 | |
| 6 | Placebo | Baseline | 8 | 32 | 100 | |
| 6 | Placebo | PreVfib | 6 | 31.5 | 100 | |
| 6 | Placebo | NoFlow | 3 | 29.6 | 100 | |
| 6 | Placebo | CPR5 | 0 | 27.9 | 99 | |
| 6 | Placebo | CPR10 | −8 | 22.7 | 86 | |
| 6 | Placebo | CPR15 | −22 | 7.1 | 90 | |
| 6 | Placebo | CPR20 | −8 | 19 | 81 | |
| 6 | Placebo | CPR25 | −25 | 4 | 68 | |
| 6 | Placebo | CPR30 | −12 | 18.9 | 59 | |
| 6 | Placebo | CPR35 | −22 | 11.1 | 66 | |
| 6 | Placebo | CPR40 | −21 | 12.6 | 61 | |
| 7 | Placebo | Baseline | 10 | 33.2 | 100 | |
| 7 | Placebo | PreVfib | 11 | 34.5 | 100 | |
| 7 | Placebo | NoFlow | | | | |
| 7 | Placebo | CPR5 | 5 | 27.3 | 100 | |
| 7 | Placebo | ROSC | −5 | 23.7 | 100 | |
| 7 | Placebo | ROSC15 | −5 | 23.6 | 100 | |
| 7 | Placebo | PreEuth | 2 | 26.8 | 100 | |
| 8 | Verum | Baseline | 9 | 32.8 | 100 | |
| 8 | Verum | PreVfib | 11 | 34.9 | 100 | |
| 8 | Verum | NoFlow | | | | |
| 8 | Verum | CPR5 | 12 | 33 | 100 | |
| 8 | Verum | CPR10 | 3 | 24.3 | 100 | |
| 8 | Verum | CPR15 | −5 | 19.8 | 100 | |
| 8 | Verum | CPR20 | −9 | 18.9 | 95 | |
| 8 | Verum | CPR25 | 22 | 47.5 | 73 | |
| 8 | Verum | CPR30 | 0 | 29.4 | 75 | |
| 8 | Verum | CPR35 | −6 | 24.2 | 76 | |
| 8 | Verum | CPR40 | −13 | 19.6 | 68 | |
| 9 | Placebo | Baseline | 13 | 36.9 | 100 | |
| 9 | Placebo | PreVfib | | | | |
| 9 | Placebo | NoFlow | 7 | 32.8 | 100 | |
| 9 | Placebo | CPR5 | 1 | 26.1 | 99 | |
| 9 | Placebo | CPR10 | −7 | 19.9 | 93 | |
| 9 | Placebo | CPR15 | −18 | 10.9 | 78 | |
| 9 | Placebo | CPR20 | −3 | 23.8 | 60 | |
| 9 | Placebo | CPR25 | | | | |
| 9 | Placebo | CPR30 | −16 | 10.9 | 74 | |
| 9 | Placebo | CPR35 | | | | |
| 9 | Placebo | CPR40 | | | | |
| 9 | Placebo | CPR45 | −10 | 22.9 | 17 | |
| 10 | Placebo | Baseline | 10 | 33.9 | 100 | |
| 10 | Placebo | PreVfib | 12 | 35.3 | 100 | |
| 10 | Placebo | NoFlow | 4 | 28.3 | 100 | |
| 10 | Placebo | ROSC | 0 | 26.1 | 100 | |
| 10 | Placebo | ROSC15 | −2 | 25.3 | 100 | |
| 10 | Placebo | ROSC30 | −1 | 26 | 100 | |
| 10 | Placebo | ROSC45 | 3 | 27.7 | 100 | |
| 11 | Verum | Baseline | 12 | 35.1 | 100 | |
| 11 | Verum | PreVfib | | | | |
| 11 | Verum | NoFlow | | | | |
| 11 | Verum | ROSC | | | | |
| 11 | Verum | ROSC15 | −1 | 25.7 | 100 | |
| 11 | Verum | ROSC30 | −5 | 23.3 | 100 | |
| 11 | Verum | ROSC45 | −2 | 24.4 | 100 | |
| 11 | Verum | ROSC60 | 0 | 26.4 | 100 | |
| 11 | Verum | ROSC75 | 2 | 30.7 | 100 | |
| 12 | Verum | Baseline | 11 | 34.8 | 100 | |
| 12 | Verum | PreVfib | 7 | 31.6 | 100 | |
| 12 | Verum | NoFlow | 3 | 28.5 | 98 | |
| 12 | Verum | CPR5 | −1 | 24.3 | 99 | |
| 12 | Verum | ROSC | −7 | 20.8 | 97 | |
| 12 | Verum | ROSC15 | −6 | 23.1 | 99 | |
| 12 | Verum | ROSC30 | −5 | 24 | 99 | |
| 12 | Verum | ROSC45 | −6 | 22.8 | 99 | |

TABLE 5
Legend for Tables 1-4

| Term | Definition |
|---|---|
| Baseline | Baseline blood gas |
| PreVfib | Before induction of ventricular fibrillation |
| NoFlow | at end of no flow time |
| CPR5 | at five minutes of CPR |
| CPR10 | at 10 minutes of CPR |
| CPR15 | at 15 minutes of CPR |
| CPR20 | at 20 minutes of CPR |
| CPR25 | at 25 minutes of CPR |
| CPR30 | at 30 minutes of CPR |
| CPR35 | at 35 minutes of CPR |
| CPR40 | at 40 minutes of CPR |
| CPR45 | at 45 minutes of CPR |
| ROSC | upon return of spontaneous circulation |
| ROSC15 | 15 minutes after ROSC |
| ROSC30 | 30 minutes after ROSC |
| ROSC45 | 45 minutes after ROSC |
| ROSC60 | 60 minutes after ROSC |
| ROSC75 | 75 minutes after ROSC |
| PreEuth | prior to euthanasia |

A general trend toward improved oxygenation as measured by sO2 (oxygen saturation), and pO2 (partial pressure of oxygen) was noted in both placebo and DDFPe groups during resuscitation. Markers of acidosis, specifically pH and pCO2 (partial pressure of carbon dioxide), also reflected an initial poorly perfused state that corrected as resuscitation progressed in survivors, though the DDFPe group tended to exhibit slightly higher pCO2 and lower pH than control animals.

Echocardiographic data demonstrated normal ejection fraction and normal regional wall motion in all animals at baseline. Two surviving animals (one placebo, one verum) demonstrated hyperdynamic ejection fraction after achieving stable ROSC. One animal in the DDFPe group demonstrated mild septal wall motion abnormality that resolved by the time of recovery from anesthesia. After ROSC, one animal in the verum group developed a pneumothorax and another in the placebo group developed pericardial effusion, both attributed to prolonged CPR. Neither was successfully recovered from anesthesia.

### Biomarker Assessment

Baseline biomarker data between DDFPe and placebo groups are shown in Table 6. To assess changes in biomarkers over time between placebo and verum groups, interactions between treatnwet and time were evaluated in linear mixed effects models with subject-specific random intercepts.

For NFL (neurofilament light chain), the interaction between treatment and time had an estimate of 0.42 (p-value <0.001). The interaction indicates a difference in the change in NFL (neurofilament light chain) level per unit of time (1 hour) between the treatment groups The DDFPe group had an average 042-unit higher increase in NFL (neurofilament light chain) as compared to the placebo group. Of the remaining biomarkers, no statistically significant interaction was noted. For GFAP. the interaction between treatment and time had an estimate of 0.41 (p-value <0.237, Tau had an estimate of 0.01 (p-value <0.137), and UCH-L1 had an estimate of32.31 (p-value <0.301).

In Table 6, Verum (bolded in treatment column), Placebo (italicized in treatment column). Column 1 (Subject Number): Column 2 (Treatment, DDFPe-treated (verum), placebo-treated (placebo)); Column 3 (hours): Column 4 (GFAP (pg/mL)), Column 5 (NFL (pg/mL)); Column 6 (Tau (pg/mL)); Column 7 (UCH-L1 (pg/mL)); Column 8 (ROSC, y (yes) or n(no)); Column 9 (Survival y (yes) or n(no)).

ROSC is defined as “stable ROSC” if sustained without significant support for 15 minutes per the protocol.

Legend for Column 3 in Table 6.
0 hours: baseline sample taken approximately 30 minutes before cardiac arrest induced
0.25 hour: for subjects 1, 2, 3, sample taken at time stable ROSC declared (after ROSC sustained without significant support for 15 minutes)
1.5, 2, 24, 48, 72, and 96 hour: samples taken at indicated time after stable ROSC declared (subjects 1-2, 4-5, and 10-12)
   For subject 6, this animal did not achieve “stable ROSC” as defined by the protocol and did not survive to coming off the ventilator, so a biomarker sample was drawn at the 2 hour mark from the time of arrest induction and animal subsequently euthanized)

TABLE 6
Biomarker Data

| Subject | | | GFAP | NFL | Tau | UCH-L1 | | |
|---|---|---|---|---|---|---|---|---|
| [symbol] | Treatment | Hours | (pg/mL) | (pg/mL) | (pg/mL) | (pg/mL) | ROSC | Survival |
| 1 | Verum | 0 | 42.01 | 13.39 | 1.22 | 2467.69 | y | y |
| 1 | Verum | 0.25 | 41.90 | 18.32 | 1.66 | 245.89 | y | y |
| 1 | Verum | 2 | | | | | | |
| 1 | Verum | 24 | 54.87 | 23.30 | 0.77 | 93.14 | y | y |
| 1 | Verum | 48 | 53.57 | 59.98 | 0.78 | 74.01 | y | y |
| 1 | Verum | 72 | 69.86 | 82.08 | 1.26 | 138.17 | y | y |
| 1 | Verum | 96 | 85.73 | 60.02 | 3.30 | 136.57 | y | y |
| 2 | Verum | 0 | 28.49 | 14.52 | 3.49 | 57.95 | y | n |
| 2 | Verum | 0.25 | 29.72 | 24.44 | 1.24 | 98.52 | y | n |
| 2 | Verum | 1.5 | 26.01 | 27.47 | 4.74 | 65.48 | y | n |
| 2 | Verum | 2 | | | | | | |
| 2 | Verum | 24 | | | | | | |
| 2 | Verum | 48 | | | | | | |
| 2 | Verum | 72 | | | | | | |
| 2 | Verum | 96 | | | | | | |
| 3 | Placebo | 0 | 27.62 | 6.52 | 1.33 | 587.51 | n | n |
| 3 | Placebo | 0.25 | 123.81 | 7.59 | 1.06 | 4458.60 | n | n |
| 3 | Placebo | 2 | | | | | | |
| 3 | Placebo | 24 | | | | | | |
| 3 | Placebo | 48 | | | | | | |
| 3 | Placebo | 72 | | | | | | |
| 3 | Placebo | 96 | | | | | | |
| 4 | Verum | 0 | 9.87 | 7.06 | 0.06 | 14.67 | y | y |
| 4 | Verum | 2 | 6.90 | 9.88 | 0.08 | 35.70 | y | y |
| 4 | Verum | 24 | 8.58 | 23.70 | 0.08 | 8.30 | y | y |
| 4 | Verum | 48 | 7.17 | 20.94 | 0.12 | 21.22 | y | y |
| 4 | Verum | 72 | 9.06 | 38.66 | 0.32 | 9.55 | y | y |
| 4 | Verum | 96 | 6.78 | 32.60 | 0.25 | | y | y |
| 5 | Verum | 0 | 11.44 | 9.49 | 0.10 | 26.38 | y | n |
| 5 | Verum | 2 | 7.02 | 8.33 | 0.01 | 97.60 | y | n |
| 5 | Verum | 24 | | | | | | |
| 5 | Verum | 48 | | | | | | |
| 5 | Verum | 72 | | | | | | |
| 5 | Verum | 96 | | | | | | |
| 6 | Placebo | 0 | 30.26 | 7.74 | 0.07 | 141.60 | n | n |
| 6 | Placebo | 2 | 4.93 | 1.74 | 0.10 | 9.96 | n | n |
| 6 | Placebo | 24 | | | | | | |
| 6 | Placebo | 48 | | | | | | |
| 6 | Placebo | 72 | | | | | | |
| 6 | Placebo | 96 | | | | | | |
| 7 | Placebo | 0 | 458.96 | 44.93 | 5.72 | 12923.37 | y | n |
| 7 | Placebo | 2 | | | | | | |
| 7 | Placebo | 24 | | | | | | |
| 7 | Placebo | 48 | | | | | | |
| 7 | Placebo | 72 | | | | | | |
| 7 | Placebo | 96 | | | | | | |
| 8 | Verum | 0 | | | | | n | n |
| 8 | Verum | 2 | | | | | | |
| 8 | Verum | 24 | | | | | | |
| 8 | Verum | 48 | | | | | | |
| 8 | Verum | 72 | | | | | | |
| 8 | Verum | 96 | | | | | | |
| 9 | Placebo | 0 | | | | | n | n |
| 9 | Placebo | 2 | | | | | | |
| 9 | Placebo | 24 | | | | | | |
| 9 | Placebo | 48 | | | | | | |
| 9 | Placebo | 72 | | | | | | |
| 9 | Placebo | 96 | | | | | | |
| 10 | Placebo | 0 | 50.67 | 25.35 | 1.23 | 136.35 | y | y |
| 10 | Placebo | 2 | 33.08 | 24.15 | 0.56 | 161.87 | y | y |
| 10 | Placebo | 24 | 43.60 | 13.93 | 0.23 | 74.94 | y | y |
| 10 | Placebo | 48 | 5.50 | 14.62 | 0.14 | 89.43 | y | y |
| 10 | Placebo | 72 | 32.04 | 15.32 | 0.18 | 64.31 | y | y |
| 10 | Placebo | 96 | 32.37 | 18.06 | 0.13 | 22.81 | y | y |
| 11 | Verum | 0 | 16.55 | 7.09 | 0.14 | 29.99 | y | y |
| 11 | Verum | 2 | 49.31 | 10.93 | 0.17 | 95.99 | y | y |
| 11 | Verum | 24 | 23.20 | 23.72 | 0.15 | 86.65 | y | y |
| 11 | Verum | 48 | 29.48 | 26.96 | 0.12 | 20.40 | y | y |
| 11 | Verum | 72 | 13.44 | 39.05 | 0.12 | 17.82 | y | y |
| 11 | Verum | 96 | 15.80 | 29.34 | 0.11 | 208.14 | y | y |
| 12 | Verum | 0 | 66.46 | 19.65 | 0.22 | 212.34 | y | y |
| 12 | Verum | 2 | 38.05 | 25.60 | 0.12 | 332.98 | y | y |
| 12 | Verum | 24 | 64.02 | 35.44 | 0.17 | 237.46 | y | y |
| 12 | Verum | 48 | 89.99 | 38.54 | 0.10 | 141.94 | y | y |
| 12 | Verum | 72 | 145.51 | 53.12 | 0.57 | 802.43 | y | y |
| 12 | Verum | 96 | 73.52 | 40.38 | 0.11 | 463.85 | y | y |

[symbol]

indicates data missing or illegible when filed

### Functional Assessment

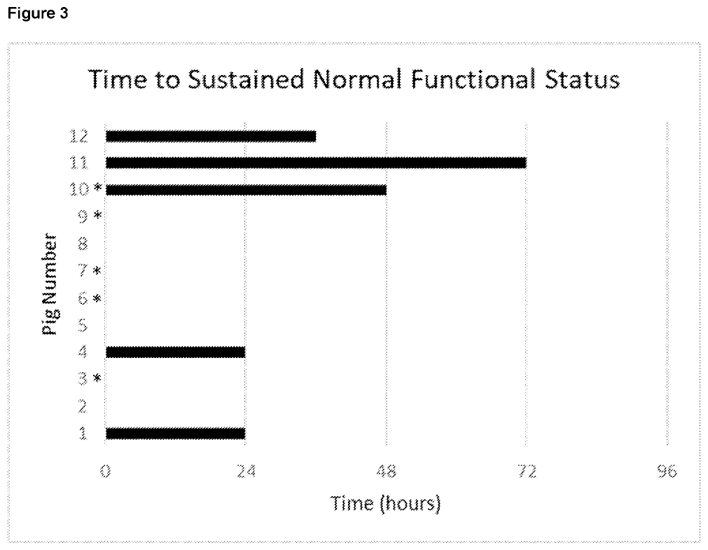

A total of 5 animals survived the 96-hour observation period; 1 placebo and 4 DDFPe. All animals had normal pre-procedure function across all neurological assessment scales. In the initial hours following resuscitation, surviving animals tended to have decreased motor responses, ataxic gait, and sluggish responses that recovered to sustained normality by 72 hours post-ROSC (FIG. 3). Average time to full recovery was 38 hours in DDFPe group and 48 hours for the sole placebo animal that survived the study period. FIG. 6 shows the functional status of animals in both placebo and DDFPe cohorts. FIG. 4 shows the progression of functional status in surviving animals. Specific functional scores at each observation time point for each animal are presented in Tables 7-12.

Legend for Column 2 (Day Number and Time (military)) in Tables 7-12:

### Days:

Day 0: Day prior to that when cardiac arrest induced
Day 1: Day cardiac arrest induced [Cardiac arrest typically induced around 10:30 military time on Day 1
Day 2: One day after day when cardiac arrest induced
Day 3: Two days after day when cardiac arrest induced
Day 4: Three days after day when cardiac arrest induced
Day 5: Four days after day when cardiac arrest induced

Table 7: Column 1 (Animal Number); Column 2 (Day Number and Time (military)); Column 3 (Overall Performance Category (OPC))

Table 8: Column 1 (Animal Number); Column 2 (Day Number and Time (military)); Column 3 (Posture); Column 4 (Gait)

Table 9: Column 1 (Animal Number); Column 2 (Day Number and Time (military)); Column 3 (Stimuli); Column 4 (Pupils); Column 5 (Convulsions)

Table 10: Column 1 (Animal Number); Column 2 (Day Number and Time (military)); Column 3 (Level of Consciousness); Column 4 (Motor response (to pinch hoof-pad))

Table 11: Column 1 (Animal Number); Column 2 (Day Number and Time (military)); Column 3 (Muscle Tone) (pick up and release extremity); Column 4 (Respiratory Pattern); Column 5 (Standing)

Table 12: Column 1 (Animal Number); Column 2 (Day Number and Time (military)); Column 3 (Walking); Column 4 (Restraint (attempt to hold down pig from behind))

TABLE 7
Functional Assessment: Overall Performance Category (OPC)

| | Day | |
|---|---|---|
| | Number | |
| Animal | and Time | |
| Number | (military) | Overall Performance Category (OPC) |
| 1 | Day 0 800 | OPC1: Normality |
| 1 | Day 1 | OPC3: Severe disability, animals neither fully aware |
| | 1350 | or unconscious, but with reaction to pain and |
| | | auditory stimuli |
| 1 | Day | OPC1: Normality |
| | 1 1830 | |
| 1 | Day | OPC1: Normality |
| | 1 2330 | |
| 1 | Day | OPC1: Normality |
| | 2 600 | |
| 1 | Day | OPC1: Normality |
| | 2 1130 | |
| 1 | Day | OPC1: Normality |
| | 2 1800 | |
| 1 | Day | OPC1: Normality |
| | 2 2359 | |
| 1 | Day | OPC1: Normality |
| | 3 600 | |
| 1 | Day | OPC1: Normality |
| | 3 1200 | |
| 1 | Day | OPC1: Normality |
| | 3 1800 | |
| 1 | Day | OPC1: Normality |
| | 3 2200 | |
| 1 | Day | OPC1: Normality |
| | 4 600 | |
| 1 | Day | OPC1: Normality |
| | 4 1200 | |
| 1 | Day | OPC1: Normality |
| | 4 1800 | |
| 1 | Day | OPC1: Normality |
| | 4 2359 | |
| 1 | Day | OPC1: Normality |
| | 5 600 | |
| 1 | Day | OPC1: Normality |
| | 5 1200 | |
| 2 | Day | OPC1: Normality |
| | 0 600 | |
| 3 | Day | OPC1: Normality |
| | 0 600 | |
| 4 | Day | OPC1: Normality |
| | 0 1816 | |
| 4 | Day | OPC1: Normality |
| | 1 2159 | |
| 4 | Day | OPC1: Normality |
| | 2 908 | |
| 4 | Day | OPC1: Normality |
| | 3 909 | |
| 4 | Day | OPC1: Normality |
| | 3 2145 | |
| 4 | Day | OPC1: Normality |
| | 4 845 | |
| 4 | Day | OPC1: Normality |
| | 5 830 | |
| 5 | Day | OPC1: Normality |
| | 0 2030 | |
| 6 | Day | OPC1: Normality |
| | 0 17:15 | |
| 7 | Day | OPC1: Normality |
| | 0 825 | |
| 7 | Day | OPC1: Normality |
| | 0 927 | |
| 7 | Day | OPC1: Normality |
| | 0 2000 | |
| 8 | Day | OPC1: Normality |
| | 0 1747 | |
| 8 | Day | OPC1: Normality |
| | 0 800 | |
| 8 | Day | OPC1: Normality |
| | 0 2039 | |
| 9 | Day | OPC1: Normality |
| | 0 656 | |
| 10 | Day | OPC1: Normality |
| | 0 650 | |
| 10 | Day | OPC1: Normality |
| | 1 1949 | |
| 10 | Day | OPC1: Normality |
| | 2 1730 | |
| 10 | Day | OPC1: Normality |
| | 3 1615 | |
| 10 | Day | OPC1: Normality |
| | 4 1400 | |
| 10 | Day | OPC1: Normality |
| | 5 1230 | |
| 11 | Day | OPC1: Normality |
| | 0 801 | |
| 11 | Day | OPC1: Normality |
| | 1 1823 | |
| 11 | Day | OPC1: Normality |
| | 2 1300 | |
| 11 | Day | OPC1: Normality |
| | 2 1852 | |
| 11 | Day | OPC1: Normality |
| | 3 1100 | |
| 11 | Day | OPC1: Normality |
| | 3 2200 | |
| 11 | Day | OPC1: Normality |
| | 4 1256 | |
| 11 | Day | OPC1: Normality |
| | 4 1820 | |
| 11 | Day | OPC1: Normality |
| | 5 1052 | |
| 12 | Day | OPC1: Normality |
| | 0 820 | |
| 12 | Day | OPC1: Normality |
| | 0 1900 | |
| 12 | Day | OPC1: Normality |
| | 1 800 | |
| 12 | Day | OPC1: Normality |
| | 1 1845 | |
| 12 | Day | OPC1: Normality |
| | 1 2000 | |
| 12 | Day | OPC1: Normality |
| | 1 2259 | |
| 12 | Day | OPC1: Normality |
| | 2 800 | |
| 12 | Day | OPC1: Normality |
| | 2 1840 | |
| 12 | Day | OPC1: Normality |
| | 3 900 | |
| 12 | Day | OPC1: Normality |
| | 4 1355 | |
| 12 | Day | OPC1: Normality |
| | 4 2101 | |
| 12 | Day | OPC1: Normality |
| | 5 1142 | |

TABLE 8
Functional Assessment: Posture, Gait

| | Day | | |
|---|---|---|---|
| | Number | | |
| Animal | and Time | | |
| Number | (military) | Posture | Gait |
| 1 | Day 0 800 | Stands normally (20 points) | Normal (20 points) |
| 1 | Day 1 | Lying on each side (0 points) | Ataxic (10 points) |
| | 1350 | | |
| 1 | Day | Stands normally (20 points) | Normal (20 points) |
| | 1 1830 | | |
| 1 | Day | Stands normally (20 points) | Normal (20 points) |
| | 1 2330 | | |
| 1 | Day | Stands normally (20 points) | Normal (20 points) |
| | 2 600 | | |
| 1 | Day | Stands normally (20 points) | Normal (20 points) |
| | 2 1130 | | |
| 1 | Day | Stands normally (20 points) | Normal (20 points) |
| | 2 1800 | | |
| 1 | Day | Stands normally (20 points) | Normal (20 points) |
| | 2 2359 | | |
| 1 | Day | Stands normally (20 points) | Normal (20 points) |
| | 3 600 | | |
| 1 | Day | Stands normally (20 points) | Normal (20 points) |
| | 3 1200 | | |
| 1 | Day | Stands normally (20 points) | Normal (20 points) |
| | 3 1800 | | |
| 1 | Day | Stands normally (20 points) | Normal (20 points) |
| | 3 2200 | | |
| 1 | Day | Stands normally (20 points) | Normal (20 points) |
| | 4 600 | | |
| 1 | Day | Stands normally (20 points) | Normal (20 points) |
| | 4 1200 | | |
| 1 | Day | Stands normally (20 points) | Normal (20 points) |
| | 4 1800 | | |
| 1 | Day | Stands normally (20 points) | Normal (20 points) |
| | 4 2359 | | |
| 1 | Day | Stands normally (20 points) | Normal (20 points) |
| | 5 600 | | |
| 1 | Day | Stands normally (20 points) | Normal (20 points) |
| | 5 1200 | | |
| 2 | Day | Stands normally (20 points) | Normal (20 points) |
| | 0 600 | | |
| 3 | Day | Stands normally (20 points) | Normal (20 points) |
| | 0 600 | | |
| 4 | Day | Stands normally (20 points) | Normal (20 points) |
| | 0 1816 | | |
| 4 | Day | Stands normally (20 points) | Normal (20 points) |
| | 1 2159 | | |
| 4 | Day | Stands normally (20 points) | None (0 points) |
| | 2 908 | | |
| 4 | Day | Stands normally (20 points) | Normal (20 points) |
| | 3 909 | | |
| 4 | Day | Stands normally (20 points) | Normal (20 points) |
| | 3 2145 | | |
| 4 | Day | Stands normally (20 points) | Normal (20 points) |
| | 4 845 | | |
| 4 | Day | Stands normally (20 points) | Normal (20 points) |
| | 5 830 | | |
| 5 | Day | Stands normally (20 points) | Normal (20 points) |
| | 0 2030 | | |
| 6 | Day | Stands normally (20 points) | Normal (20 points) |
| | 0 17:15 | | |
| 7 | Day | Stands normally (20 points) | Normal (20 points) |
| | 0 825 | | |
| 7 | Day | Stands normally (20 points) | Normal (20 points) |
| | 0 927 | | |
| 7 | Day | Stands normally (20 points) | Normal (20 points) |
| | 0 2000 | | |
| 8 | Day | Stands normally (20 points) | Normal (20 points) |
| | 0 1747 | | |
| 8 | Day | Stands normally (20 points) | Normal (20 points) |
| | 0 800 | | |
| 8 | Day | Stands normally (20 points) | Normal (20 points) |
| | 0 2039 | | |
| 9 | Day | Stands normally (20 points) | Normal (20 points) |
| | 0 656 | | |
| 10 | Day | Stands normally (20 points) | Normal (20 points) |
| | 0 650 | | |
| 10 | Day | Stands normally (20 points) | Ataxic (10 points) |
| | 1 1949 | | |
| 10 | Day | Stands normally (20 points) | Normal (20 points) |
| | 2 1730 | | |
| 10 | Day | Stands normally (20 points) | Normal (20 points) |
| | 3 1615 | | |
| 10 | Day | Stands normally (20 points) | Normal (20 points) |
| | 4 1400 | | |
| 10 | Day | Stands normally (20 points) | Normal (20 points) |
| | 5 1230 | | |
| 11 | Day | Stands normally (20 points) | Normal (20 points) |
| | 0 801 | | |
| 11 | Day | Stands normally (20 points) | Normal (20 points) |
| | 1 1823 | | |
| 11 | Day | Stands normally (20 points) | Normal (20 points) |
| | 2 1300 | | |
| 11 | Day | Stands normally (20 points) | Normal (20 points) |
| | 2 1852 | | |
| 11 | Day | Stands normally (20 points) | Normal (20 points) |
| | 3 1100 | | |
| 11 | Day | Stands normally (20 points) | Normal (20 points) |
| | 3 2200 | | |
| 11 | Day | Stands normally (20 points) | Normal (20 points) |
| | 4 1256 | | |
| 11 | Day | Stands normally (20 points) | Normal (20 points) |
| | 4 1820 | | |
| 11 | Day | Stands normally (20 points) | Normal (20 points) |
| | 5 1052 | | |
| 12 | Day | Stands normally (20 points) | Normal (20 points) |
| | 0 820 | | |
| 12 | Day | Stands normally (20 points) | Normal (20 points) |
| | 0 1900 | | |
| 12 | Day | Stands normally (20 points) | Normal (20 points) |
| | 1 800 | | |
| 12 | Day | Stands normally (20 points) | Normal (20 points) |
| | 1 1845 | | |
| 12 | Day | Stands normally (20 points) | Ataxic (10 points) |
| | 1 2000 | | |
| 12 | Day | Stands normally (20 points) | Normal (20 points) |
| | 1 2259 | | |
| 12 | Day | Stands normally (20 points) | Normal (20 points) |
| | 2 800 | | |
| 12 | Day | Stands normally (20 points) | Normal (20 points) |
| | 2 1840 | | |
| 12 | Day | Stands normally (20 points) | Normal (20 points) |
| | 3 900 | | |
| 12 | Day | Stands normally (20 points) | Normal (20 points) |
| | 4 1355 | | |
| 12 | Day | Stands normally (20 points) | Normal (20 points) |
| | 4 2101 | | |
| 12 | Day | Stands normally (20 points) | Normal (20 points) |
| | 5 1142 | | |

TABLE 9
Functional Assessment: Stimuli, Pupils, Convulsions

| | Day | | | |
|---|---|---|---|---|
| | Number | | | |
| Animal | and Time | | | |
| Number | (military) | Stimuli | Pupils | Convulsions |
| 1 | Day 0 800 | Response to all | Normal | None |
| | | stimuli (20 points) | (20 points) | (20 points) |
| 1 | Day 1 1350 | Response to all | Mydriasis (0 | None |
| | | stimuli (20 points) | points) | (20 points) |
| 1 | Day | Response to all | Normal | None |
| | 1 1830 | stimuli (20 points) | (20 points) | (20 points) |
| 1 | Day | Response to all | Normal | None |
| | 1 2330 | stimuli (20 points) | (20 points) | (20 points) |
| 1 | Day 2 600 | Response to all | Normal | None |
| | | stimuli (20 points) | (20 points) | (20 points) |
| 1 | Day | Response to all | Normal | None |
| | 2 1130 | stimuli (20 points) | (20 points) | (20 points) |
| 1 | Day | Response to all | Normal | None |
| | 2 1800 | stimuli (20 points) | (20 points) | (20 points) |
| 1 | Day | Response to all | Normal | None |
| | 2 2359 | stimuli (20 points) | (20 points) | (20 points) |
| 1 | Day 3 600 | Response to all | Normal | None |
| | | stimuli (20 points) | (20 points) | (20 points) |
| 1 | Day | Response to all | Normal | None |
| | 3 1200 | stimuli (20 points) | (20 points) | (20 points) |
| 1 | Day | Response to all | Normal | None |
| | 3 1800 | stimuli (20 points) | (20 points) | (20 points) |
| 1 | Day | Response to all | Normal | None |
| | 3 2200 | stimuli (20 points) | (20 points) | (20 points) |
| 1 | Day 4 600 | Response to all | Normal | None |
| | | stimuli (20 points) | (20 points) | (20 points) |
| 1 | Day | Response to all | Normal | None |
| | 4 1200 | stimuli (20 points) | (20 points) | (20 points) |
| 1 | Day | Response to all | Normal | None |
| | 4 1800 | stimuli (20 points) | (20 points) | (20 points) |
| 1 | Day | Response to all | Normal | None |
| | 4 2359 | stimuli (20 points) | (20 points) | (20 points) |
| 1 | Day 5 600 | Response to all | Normal | None |
| | | stimuli (20 points) | (20 points) | (20 points) |
| 1 | Day | Response to all | Normal | None |
| | 5 1200 | stimuli (20 points) | (20 points) | (20 points) |
| 2 | Day 0 600 | Response to all | Normal | None |
| | | stimuli (20 points) | (20 points) | (20 points) |
| 3 | Day 0 600 | Response to all | Normal | None |
| | | stimuli (20 points) | (20 points) | (20 points) |
| 4 | Day | Response to all | Normal | None |
| | 0 1816 | stimuli (20 points) | (20 points) | (20 points) |
| 4 | Day | Response to all | Normal | None |
| | 1 2159 | stimuli (20 points) | (20 points) | (20 points) |
| 4 | Day 2 908 | Response to all | Normal | None |
| | | stimuli (20 points) | (20 points) | (20 points) |
| 4 | Day 3 909 | Response to all | Normal | None |
| | | stimuli (20 points) | (20 points) | (20 points) |
| 4 | Day | Response to all | Normal | None |
| | 3 2145 | stimuli (20 points) | (20 points) | (20 points) |
| 4 | Day 4 845 | Response to all | Normal | None |
| | | stimuli (20 points) | (20 points) | (20 points) |
| 4 | Day 5 830 | Response to all | Normal | None |
| | | stimuli (20 points) | (20 points) | (20 points) |
| 5 | Day | Response to all | Normal | None |
| | 0 2030 | stimuli (20 points) | (20 points) | (20 points) |
| 6 | Day | Response to all | Normal | None |
| | 0 17:15 | stimuli (20 points) | (20 points) | (20 points) |
| 7 | Day 0 825 | Response to all | Normal | None |
| | | stimuli (20 points) | (20 points) | (20 points) |
| 7 | Day 0 927 | Response to all | Normal | None |
| | | stimuli (20 points) | (20 points) | (20 points) |
| 7 | Day | Response to all | Normal | None |
| | 0 2000 | stimuli (20 points) | (20 points) | (20 points) |
| 8 | Day | Response to all | Normal | None |
| | 0 1747 | stimuli (20 points) | (20 points) | (20 points) |
| 8 | Day 0 800 | Response to all | Normal | None |
| | | stimuli (20 points) | (20 points) | (20 points) |
| 8 | Day | Response to all | Normal | None |
| | 0 2039 | stimuli (20 points) | (20 points) | (20 points) |
| 9 | Day 0 656 | Response to all | Normal | None |
| | | stimuli (20 points) | (20 points) | (20 points) |
| 10 | Day 0 650 | Response to all | Normal | None |
| | | stimuli (20 points) | (20 points) | (20 points) |
| 10 | Day | Response to all | Normal | None |
| | 1 1949 | stimuli (20 points) | (20 points) | (20 points) |
| 10 | Day | Response to all | Normal | None |
| | 2 1730 | stimuli (20 points) | (20 points) | (20 points) |
| 10 | Day | Response to all | Normal | None |
| | 3 1615 | stimuli (20 points) | (20 points) | (20 points) |
| 10 | Day | Response to all | Normal | None |
| | 4 1400 | stimuli (20 points) | (20 points) | (20 points) |
| 10 | Day | Response to all | Normal | None |
| | 5 1230 | stimuli (20 points) | (20 points) | (20 points) |
| 11 | Day 0 801 | Response to all | Normal | None |
| | | stimuli (20 points) | (20 points) | (20 points) |
| 11 | Day | No response (0 | Normal | None |
| | 1 1823 | points) | (20 points) | (20 points) |
| 11 | Day | Response to all | Normal | None |
| | 2 1300 | stimuli (20 points) | (20 points) | (20 points) |
| 11 | Day | Response to all | Normal | None |
| | 2 1852 | stimuli (20 points) | (20 points) | (20 points) |
| 11 | Day | Response to all | Normal | None |
| | 3 1100 | stimuli (20 points) | (20 points) | (20 points) |
| 11 | Day | Response to all | Normal | None |
| | 3 2200 | stimuli (20 points) | (20 points) | (20 points) |
| 11 | Day | Response to all | Normal | None |
| | 4 1256 | stimuli (20 points) | (20 points) | (20 points) |
| 11 | Day | Response to all | Normal | None |
| | 4 1820 | stimuli (20 points) | (20 points) | (20 points) |
| 11 | Day | Response to all | Normal | None |
| | 5 1052 | stimuli (20 points) | (20 points) | (20 points) |
| 12 | Day 0 820 | Response to all | Normal | None |
| | | stimuli (20 points) | (20 points) | (20 points) |
| 12 | Day | Response to all | Normal | None |
| | 0 1900 | stimuli (20 points) | (20 points) | (20 points) |
| 12 | Day 1 800 | Response to all | Normal | None |
| | | stimuli (20 points) | (20 points) | (20 points) |
| 12 | Day | Response to all | Normal | None |
| | 1 1845 | stimuli (20 points) | (20 points) | (20 points) |
| 12 | Day | Response to all | Normal | None |
| | 1 2000 | stimuli (20 points) | (20 points) | (20 points) |
| 12 | Day | Response to all | Normal | None |
| | 1 2259 | stimuli (20 points) | (20 points) | (20 points) |
| 12 | Day 2 800 | Response to all | Normal | None |
| | | stimuli (20 points) | (20 points) | (20 points) |
| 12 | Day | Response to all | Normal | None |
| | 2 1840 | stimuli (20 points) | (20 points) | (20 points) |
| 12 | Day 3 900 | Response to all | Normal | None |
| | | stimuli (20 points) | (20 points) | (20 points) |
| 12 | Day | Response to all | Normal | None |
| | 4 1355 | stimuli (20 points) | (20 points) | (20 points) |
| 12 | Day | Response to all | Normal | None |
| | 4 2101 | stimuli (20 points) | (20 points) | (20 points) |
| 12 | Day | Response to all | Normal | None |
| | 5 1142 | stimuli (20 points) | (20 points) | (20 points) |

TABLE 10
Functional Assessment: Level of Consciousness,
Motor response (to pinch hoof pad)

| | Day | | |
|---|---|---|---|
| | Number | | |
| | and | | Motor response |
| Animal | Time | | (to pinch |
| Number | (military) | Level of Consciousness | hoof-pad) |
| 1 | Day 0 800 | 0- Normal: complete | 0- Normal: brisk |
| | | awareness of | withdrawal |
| | | auditory stimuli | |
| 1 | Day 1 | 30- Clouded: conscious, | 0- Normal: brisk |
| | 1350 | but drowsy or irritable | withdrawal |
| 1 | Day | 30- Clouded: conscious, | 0- Normal: brisk |
| | 1 1830 | but drowsy or irritable | withdrawal |
| 1 | Day | 0- Normal: complete | 0- Normal: brisk |
| | 1 2330 | awareness of | withdrawal |
| | | auditory stimuli | |
| 1 | Day | 0- Normal: complete | 0- Normal: brisk |
| | 2 600 | awareness of | withdrawal |
| | | auditory stimuli | |
| 1 | Day | 0- Normal: complete | 0- Normal: brisk |
| | 2 1130 | awareness of | withdrawal |
| | | auditory stimuli | |
| 1 | Day | 0- Normal: complete | 0- Normal: brisk |
| | 2 1800 | awareness of | withdrawal |
| | | auditory stimuli | |
| 1 | Day | 0- Normal: complete | 0- Normal: brisk |
| | 2 2359 | awareness of | withdrawal |
| | | auditory stimuli | |
| 1 | Day | 0- Normal: complete | 0- Normal: brisk |
| | 3 600 | awareness of | withdrawal |
| | | auditory stimuli | |
| 1 | Day | 0- Normal: complete | 0- Normal: brisk |
| | 3 1200 | awareness of | withdrawal |
| | | auditory stimuli | |
| 1 | Day | 0- Normal: complete | 0- Normal: brisk |
| | 3 1800 | awareness of | withdrawal |
| | | auditory stimuli | |
| 1 | Day | 0- Normal: complete | 0- Normal: brisk |
| | 3 2200 | awareness of | withdrawal |
| | | auditory stimuli | |
| 1 | Day | 0- Normal: complete | 0- Normal: brisk |
| | 4 600 | awareness of | withdrawal |
| | | auditory stimuli | |
| 1 | Day | 0- Normal: complete | 0- Normal: brisk |
| | 4 1200 | awareness of | withdrawal |
| | | auditory stimuli | |
| 1 | Day | 0- Normal: complete | 0- Normal: brisk |
| | 4 1800 | awareness of | withdrawal |
| | | auditory stimuli | |
| 1 | Day | 0- Normal: complete | 0- Normal: brisk |
| | 4 2359 | awareness of | withdrawal |
| | | auditory stimuli | |
| 1 | Day | 0- Normal: complete | 0- Normal: brisk |
| | 5 600 | awareness of | withdrawal |
| | | auditory stimuli | |
| 1 | Day | 0- Normal: complete | 0- Normal: brisk |
| | 5 1200 | awareness of | withdrawal |
| | | auditory stimuli | |
| 2 | Day | 0- Normal: complete | 0- Normal: brisk |
| | 0 600 | awareness of | withdrawal |
| | | auditory stimuli | |
| 3 | Day | 0- Normal: complete | 0- Normal: brisk |
| | 0 600 | awareness of | withdrawal |
| | | auditory stimuli | |
| 4 | Day | 0- Normal: complete | 0- Normal: brisk |
| | 0 1816 | awareness of | withdrawal |
| | | auditory stimuli | |
| 4 | Day | 0- Normal: complete | 10- Sluggish |
| | 1 2159 | awareness of | response |
| | | auditory stimuli | |
| 4 | Day | 0- Normal: complete | 0- Normal: brisk |
| | 2 908 | awareness of | withdrawal |
| | | auditory stimuli | |
| 4 | Day | 0- Normal: complete | 0- Normal: brisk |
| | 3 909 | awareness of | withdrawal |
| | | auditory stimuli | |
| 4 | Day | 0- Normal: complete | 0- Normal: brisk |
| | 3 2145 | awareness of | withdrawal |
| | | auditory stimuli | |
| 4 | Day | 0- Normal: complete | 0- Normal: brisk |
| | 4 845 | awareness of | withdrawal |
| | | auditory stimuli | |
| 4 | Day | 0- Normal: complete | 0- Normal: brisk |
| | 5 830 | awareness of | withdrawal |
| | | auditory stimuli | |
| 5 | Day | 0- Normal: complete | 0- Normal: brisk |
| | 0 2030 | awareness of | withdrawal |
| | | auditory stimuli | |
| 6 | Day | 0- Normal: complete | 0- Normal: brisk |
| | 0 17:15 | awareness of | withdrawal |
| | | auditory stimuli | |
| 7 | Day | 0- Normal: complete | 0- Normal: brisk |
| | 0 825 | awareness of | withdrawal |
| | | auditory stimuli | |
| 7 | Day | 0- Normal: complete | 0- Normal: brisk |
| | 0 927 | awareness of | withdrawal |
| | | auditory stimuli | |
| 7 | Day | 0- Normal: complete | 0- Normal: brisk |
| | 0 2000 | awareness of | withdrawal |
| | | auditory stimuli | |
| 8 | Day | 0- Normal: complete | 0- Normal: brisk |
| | 0 1747 | awareness of | withdrawal |
| | | auditory stimuli | |
| 8 | Day | 0- Normal: complete | 0- Normal: brisk |
| | 0 800 | awareness of | withdrawal |
| | | auditory stimuli | |
| 8 | Day | 0- Normal: complete | 0- Normal: brisk |
| | 0 2039 | awareness of | withdrawal |
| | | auditory stimuli | |
| 9 | Day | 0- Normal: complete | 0- Normal: brisk |
| | 0 656 | awareness of | withdrawal |
| | | auditory stimuli | |
| 10 | Day | 0- Normal: complete | 0- Normal: brisk |
| | 0 650 | awareness of | withdrawal |
| | | auditory stimuli | |
| 10 | Day | 30- Clouded: conscious, | 10- Sluggish |
| | 1 1949 | but drowsy or irritable | response |
| 10 | Day | 0- Normal: complete | 0- Normal: brisk |
| | 2 1730 | awareness of | withdrawal |
| | | auditory stimuli | |
| 10 | Day | 0- Normal: complete | 0- Normal: brisk |
| | 3 1615 | awareness of | withdrawal |
| | | auditory stimuli | |
| 10 | Day | 0- Normal: complete | 0- Normal: brisk |
| | 4 1400 | awareness of | withdrawal |
| | | auditory stimuli | |
| 10 | Day | 0- Normal: complete | 0- Normal: brisk |
| | 5 1230 | awareness of | withdrawal |
| | | auditory stimuli | |
| 11 | Day | 0- Normal: complete | 0- Normal: brisk |
| | 0 801 | awareness of | withdrawal |
| | | auditory stimuli | |
| 11 | Day | 0- Normal: complete | 10- Sluggish |
| | 1 1823 | awareness of | response |
| | | auditory stimuli | |
| 11 | Day | 0- Normal: complete | 0- Normal: brisk |
| | 2 1300 | awareness of | withdrawal |
| | | auditory stimuli | |
| 11 | Day | 0- Normal: complete | 0- Normal: brisk |
| | 2 1852 | awareness of | withdrawal |
| | | auditory stimuli | |
| 11 | Day | 0- Normal: complete | 0- Normal: brisk |
| | 3 1100 | awareness of | withdrawal |
| | | auditory stimuli | |
| 11 | Day | 0- Normal: complete | 0- Normal: brisk |
| | 3 2200 | awareness of | withdrawal |
| | | auditory stimuli | |
| 11 | Day | 0- Normal: complete | 0- Normal: brisk |
| | 4 1256 | awareness of | withdrawal |
| | | auditory stimuli | |
| 11 | Day | 0- Normal: complete | 0- Normal: brisk |
| | 4 1820 | awareness of | withdrawal |
| | | auditory stimuli | |
| 11 | Day | 0- Normal: complete | 0- Normal: brisk |
| | 5 1052 | awareness of | withdrawal |
| | | auditory stimuli | |
| 12 | Day | 0- Normal: complete | 0- Normal: brisk |
| | 0 820 | awareness of | withdrawal |
| | | auditory stimuli | |
| 12 | Day | 0- Normal: complete | 0- Normal: brisk |
| | 0 1900 | awareness of | withdrawal |
| | | auditory stimuli | |
| 12 | Day | 0- Normal: complete | 0- Normal: brisk |
| | 1 800 | awareness of | withdrawal |
| | | auditory stimuli | |
| 12 | Day | 0- Normal: complete | 0- Normal: brisk |
| | 1 1845 | awareness of | withdrawal |
| | | auditory stimuli | |
| 12 | Day | 30- Clouded: conscious, | 10- Sluggish |
| | 1 2000 | but drowsy or irritable | response |
| 12 | Day | 0- Normal: complete | 10- Sluggish |
| | 1 2259 | awareness of | response |
| | | auditory stimuli | |
| 12 | Day | 30- Clouded: conscious, | 0- Normal: brisk |
| | 2 800 | but drowsy or irritable | withdrawal |
| 12 | Day | 0- Normal: complete | 0- Normal: brisk |
| | 2 1840 | awareness of | withdrawal |
| | | auditory stimuli | |
| 12 | Day | 0- Normal: complete | 0- Normal: brisk |
| | 3 900 | awareness of | withdrawal |
| | | auditory stimuli | |
| 12 | Day | 0- Normal: complete | 0- Normal: brisk |
| | 4 1355 | awareness of | withdrawal |
| | | auditory stimuli | |
| 12 | Day | 0- Normal: complete | 0- Normal: brisk |
| | 4 2101 | awareness of | withdrawal |
| | | auditory stimuli | |
| 12 | Day | 0- Normal: complete | 0- Normal: brisk |
| | 5 1142 | awareness of | withdrawal |
| | | auditory stimuli | |

TABLE 11
Functional Assessment: Muscle Tone (pick up and
release extremity), Respiratory Pattern, Standing

| | Day | Muscle Tone | | |
|---|---|---|---|---|
| | Number | (pick up and | | |
| Animal | and Time | release | Respiratory | |
| Number | (military) | extremity) | Pattern | Standing |
| 1 | Day 0 800 | 0- Normal | 0- Normal | 0- Can stand |
| 1 | Day 1 | 0- Normal | 50- Abnormal | 20- Cannot stand |
| | 1350 | | spontaneous | |
| | | | breathing | |
| 1 | Day | 0- Normal | 0- Normal | 0- Can stand |
| | 1 1830 | | | |
| 1 | Day | 0- Normal | 0- Normal | 0- Can stand |
| | 1 2330 | | | |
| 1 | Day | 0- Normal | 0- Normal | 0- Can stand |
| | 2 600 | | | |
| 1 | Day | 0- Normal | 0- Normal | 0- Can stand |
| | 2 1130 | | | |
| 1 | Day | 0- Normal | 0- Normal | 0- Can stand |
| | 2 1800 | | | |
| 1 | Day | 0- Normal | 0- Normal | 0- Can stand |
| | 2 2359 | | | |
| 1 | Day | 0- Normal | 0- Normal | 0- Can stand |
| | 3 600 | | | |
| 1 | Day | 0- Normal | 0- Normal | 0- Can stand |
| | 3 1200 | | | |
| 1 | Day | 0- Normal | 0- Normal | 0- Can stand |
| | 3 1800 | | | |
| 1 | Day | 0- Normal | 0- Normal | 0- Can stand |
| | 3 2200 | | | |
| 1 | Day | 0- Normal | 0- Normal | 0- Can stand |
| | 4 600 | | | |
| 1 | Day | 0- Normal | 0- Normal | 0- Can stand |
| | 4 1200 | | | |
| 1 | Day | 0- Normal | 0- Normal | 0- Can stand |
| | 4 1800 | | | |
| 1 | Day | 0- Normal | 0- Normal | 0- Can stand |
| | 4 2359 | | | |
| 1 | Day | 0- Normal | 0- Normal | 0- Can stand |
| | 5 600 | | | |
| 1 | Day | 0- Normal | 0- Normal | 0- Can stand |
| | 5 1200 | | | |
| 2 | Day | 0- Normal | 0- Normal | 0- Can stand |
| | 0 600 | | | |
| 3 | Day | 0- Normal | 0- Normal | 0- Can stand |
| | 0 600 | | | |
| 4 | Day | 0- Normal | 0- Normal | 0- Can stand |
| | 0 1816 | | | |
| 4 | Day | 0- Normal | 0- Normal | 0- Can stand |
| | 1 2159 | | | |
| 4 | Day | | 0- Normal | 0- Can stand |
| | 2 908 | | | |
| 4 | Day | 0- Normal | 0- Normal | 0- Can stand |
| | 3 909 | | | |
| 4 | Day | 0- Normal | 0- Normal | 0- Can stand |
| | 3 2145 | | | |
| 4 | Day | 0- Normal | 0- Normal | 0- Can stand |
| | 4 845 | | | |
| 4 | Day | 0- Normal | 0- Normal | 0- Can stand |
| | 5 830 | | | |
| 5 | Day | 0- Normal | 0- Normal | 0- Can stand |
| | 0 2030 | | | |
| 6 | Day | 0- Normal | 0- Normal | 0- Can stand |
| | 0 17:15 | | | |
| 7 | Day | 0- Normal | 0- Normal | 0- Can stand |
| | 0 825 | | | |
| 7 | Day | 0- Normal | 0- Normal | 0- Can stand |
| | 0 927 | | | |
| 7 | Day | 0- Normal | 0- Normal | 0- Can stand |
| | 0 2000 | | | |
| 8 | Day | 0- Normal | 0- Normal | 0- Can stand |
| | 0 1747 | | | |
| 8 | Day | 0- Normal | 0- Normal | 0- Can stand |
| | 0 800 | | | |
| 8 | Day | 0- Normal | 0- Normal | 0- Can stand |
| | 0 2039 | | | |
| 9 | Day | 0- Normal | 0- Normal | 0- Can stand |
| | 0 656 | | | |
| 10 | Day | 0- Normal | 0- Normal | 0- Can stand |
| | 0 650 | | | |
| 10 | Day | 0- Normal | 0- Normal | 0- Can stand |
| | 1 1949 | | | |
| 10 | Day | 0- Normal | 0- Normal | 0- Can stand |
| | 2 1730 | | | |
| 10 | Day | 0- Normal | 0- Normal | 0- Can stand |
| | 3 1615 | | | |
| 10 | Day | 0- Normal | 0- Normal | 0- Can stand |
| | 4 1400 | | | |
| 10 | Day | 0- Normal | 0- Normal | 0- Can stand |
| | 5 1230 | | | |
| 11 | Day | 0- Normal | 0- Normal | 0- Can stand |
| | 0 801 | | | |
| 11 | Day | 0- Normal | 50- Abnormal | 0- Can stand |
| | 1 1823 | | spontaneous | |
| | | | breathing | |
| 11 | Day | 0- Normal | 0- Normal | 0- Can stand |
| | 2 1300 | | | |
| 11 | Day | 0- Normal | 0- Normal | 0- Can stand |
| | 2 1852 | | | |
| 11 | Day | 0- Normal | 0- Normal | 0- Can stand |
| | 3 1100 | | | |
| 11 | Day | 0- Normal | 50- Abnormal | 0- Can stand |
| | 3 2200 | | spontaneous | |
| | | | breathing | |
| 11 | Day | 0- Normal | 0- Normal | 0- Can stand |
| | 4 1256 | | | |
| 11 | Day | 0- Normal | 0- Normal | 0- Can stand |
| | 4 1820 | | | |
| 11 | Day | 0- Normal | 0- Normal | 0- Can stand |
| | 5 1052 | | | |
| 12 | Day | 0- Normal | 0- Normal | 0- Can stand |
| | 0 820 | | | |
| 12 | Day | 0- Normal | 0- Normal | 0- Can stand |
| | 0 1900 | | | |
| 12 | Day | 0- Normal | 0- Normal | 0- Can stand |
| | 1 800 | | | |
| 12 | Day | 0- Normal | 0- Normal | 0- Can stand |
| | 1 1845 | | | |
| 12 | Day | 0- Normal | 0- Normal | 0- Can stand |
| | 1 2000 | | | |
| 12 | Day | 0- Normal | 50- Abnormal | 0- Can stand |
| | 1 2259 | | spontaneous | |
| | | | breathing | |
| 12 | Day | 0- Normal | 0- Normal | 0- Can stand |
| | 2 800 | | | |
| 12 | Day | 0- Normal | 0- Normal | 0- Can stand |
| | 2 1840 | | | |
| 12 | Day | 0- Normal | 0- Normal | 0- Can stand |
| | 3 900 | | | |
| 12 | Day | 0- Normal | 0- Normal | 0- Can stand |
| | 4 1355 | | | |
| 12 | Day | 0- Normal | 0- Normal | 0- Can stand |
| | 4 2101 | | | |
| 12 | Day | 0- Normal | 0- Normal | 0- Can stand |
| | 5 1142 | | | |

TABLE 12
Functional Assessment: Walking, Restraint
(attempt to hold down pig from behind)

| | Day | | |
|---|---|---|---|
| | Number | | |
| Animal | and Time | | Restraint (attempt to hold |
| Number | (military) | Walking | down pig from behind) |
| 1 | Day 0 800 | 0- Normal | 0- Normal: vigorously resists |
| 1 | Day 1 | 20- Very unsteady | 20- Sluggish: resists |
| | 1350 | or ataxic gait | |
| 1 | Day | 10- Unsteady gait | 0- Normal: vigorously resists |
| | 1 1830 | | |
| 1 | Day | 0- Normal | 0- Normal: vigorously resists |
| | 1 2330 | | |
| 1 | Day | 0- Normal | 0- Normal: vigorously resists |
| | 2 600 | | |
| 1 | Day | 0- Normal | 0- Normal: vigorously resists |
| | 2 1130 | | |
| 1 | Day | 0- Normal | 0- Normal: vigorously resists |
| | 2 1800 | | |
| 1 | Day | 0- Normal | 0- Normal: vigorously resists |
| | 2 2359 | | |
| 1 | Day | 0- Normal | 0- Normal: vigorously resists |
| | 3 600 | | |
| 1 | Day | 0- Normal | 0- Normal: vigorously resists |
| | 3 1200 | | |
| 1 | Day | 0- Normal | 0- Normal: vigorously resists |
| | 3 1800 | | |
| 1 | Day | 0- Normal | 0- Normal: vigorously resists |
| | 3 2200 | | |
| 1 | Day | 0- Normal | 0- Normal: vigorously resists |
| | 4 600 | | |
| 1 | Day | 0- Normal | 0- Normal: vigorously resists |
| | 4 1200 | | |
| 1 | Day | 0- Normal | 0- Normal: vigorously resists |
| | 4 1800 | | |
| 1 | Day | 0- Normal | 0- Normal: vigorously resists |
| | 4 2359 | | |
| 1 | Day | 0- Normal | 0- Normal: vigorously resists |
| | 5 600 | | |
| 1 | Day | 0- Normal | 0- Normal: vigorously resists |
| | 5 1200 | | |
| 2 | Day | 0- Normal | 0- Normal: vigorously resists |
| | 0 600 | | |
| 3 | Day | 0- Normal | 0- Normal: vigorously resists |
| | 0 600 | | |
| 4 | Day | 0- Normal | 0- Normal: vigorously resists |
| | 0 1816 | | |
| 4 | Day | 0- Normal | 40- Very sluggish: resists |
| | 1 2159 | | minimally |
| 4 | Day | 0- Normal | |
| | 2 908 | | |
| 4 | Day | 0- Normal | 0- Normal: vigorously resists |
| | 3 909 | | |
| 4 | Day | 0- Normal | 0- Normal: vigorously resists |
| | 3 2145 | | |
| 4 | Day | 0- Normal | 0- Normal: vigorously resists |
| | 4 845 | | |
| 4 | Day | 0- Normal | 0- Normal: vigorously resists |
| | 5 830 | | |
| 5 | Day | 0- Normal | 0- Normal: vigorously resists |
| | 0 2030 | | |
| 6 | Day | 0- Normal | 0- Normal: vigorously resists |
| | 0 17:15 | | |
| 7 | Day | 0- Normal | 0- Normal: vigorously resists |
| | 0 825 | | |
| 7 | Day | 0- Normal | 0- Normal: vigorously resists |
| | 0 927 | | |
| 7 | Day | 0- Normal | 0- Normal: vigorously resists |
| | 0 2000 | | |
| 8 | Day | 0- Normal | 0- Normal: vigorously resists |
| | 0 1747 | | |
| 8 | Day | 0- Normal | 0- Normal: vigorously resists |
| | 0 800 | | |
| 8 | Day | 0- Normal | 0- Normal: vigorously resists |
| | 0 2039 | | |
| 9 | Day | 0- Normal | 0- Normal: vigorously resists |
| | 0 656 | | |
| 10 | Day | 0- Normal | 0- Normal: vigorously resists |
| | 0 650 | | |
| 10 | Day | 10- Unsteady gait | 20- Sluggish: resists |
| | 1 1949 | | |
| 10 | Day | 0- Normal | 0- Normal: vigorously resists |
| | 2 1730 | | |
| 10 | Day | 0- Normal | 0- Normal: vigorously resists |
| | 3 1615 | | |
| 10 | Day | 0- Normal | 0- Normal: vigorously resists |
| | 4 1400 | | |
| 10 | Day | 0- Normal | 0- Normal: vigorously resists |
| | 5 1230 | | |
| 11 | Day | 0- Normal | 0- Normal: vigorously resists |
| | 0 801 | | |
| 11 | Day | 10- Unsteady gait | 20- Sluggish: resists |
| | 1 1823 | | |
| 11 | Day | 0- Normal | 0- Normal: vigorously resists |
| | 2 1300 | | |
| 11 | Day | 0- Normal | 0- Normal: vigorously resists |
| | 2 1852 | | |
| 11 | Day | 0- Normal | 0- Normal: vigorously resists |
| | 3 1100 | | |
| 11 | Day | 0- Normal | 0- Normal: vigorously resists |
| | 3 2200 | | |
| 11 | Day | 0- Normal | 0- Normal: vigorously resists |
| | 4 1256 | | |
| 11 | Day | 0- Normal | 0- Normal: vigorously resists |
| | 4 1820 | | |
| 11 | Day | 0- Normal | 0- Normal: vigorously resists |
| | 5 1052 | | |
| 12 | Day | 0- Normal | 0- Normal: vigorously resists |
| | 0 820 | | |
| 12 | Day | 0- Normal | 0- Normal: vigorously resists |
| | 0 1900 | | |
| 12 | Day | 0- Normal | 0- Normal: vigorously resists |
| | 1 800 | | |
| 12 | Day | 0- Normal | 0- Normal: vigorously resists |
| | 1 1845 | | |
| 12 | Day | 10- Unsteady gait | 20- Sluggish; resists |
| | 1 2000 | | |
| 12 | Day | 10- Unsteady gait | 20- Sluggish: resists |
| | 1 2259 | | |
| 12 | Day | 0- Normal | 0- Normal: vigorously resists |
| | 2 800 | | |
| 12 | Day | 0- Normal | 0- Normal: vigorously resists |
| | 2 1840 | | |
| 12 | Day | 0- Normal | 0- Normal: vigorously resists |
| | 3 900 | | |
| 12 | Day | 0- Normal | 0- Normal: vigorously resists |
| | 4 1355 | | |
| 12 | Day | 0- Normal | 0- Normal: vigorously resists |
| | 4 2101 | | |
| 12 | Day | 0- Normal | 0- Normal: vigorously resists |
| | 5 1142 | | |

### Histopathology

Hypoxic-ischemic brain injury typically causes scattered neuronal damage in the cortex and hippocampus following the activation of astrocytes around the damaged area.

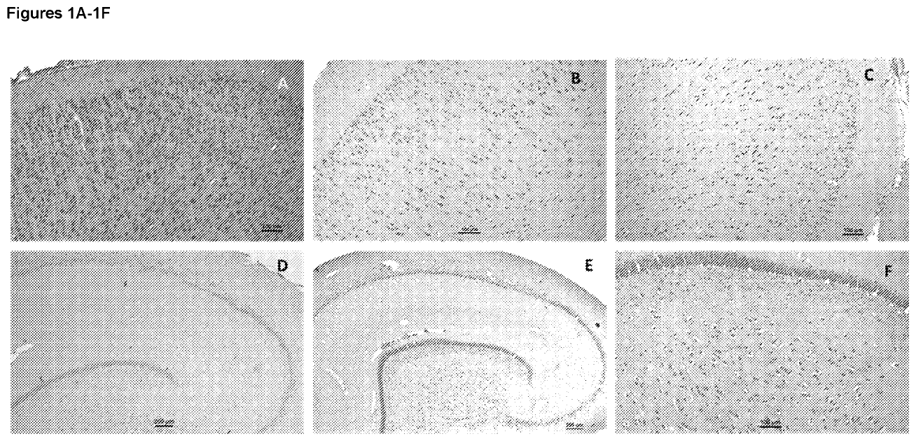

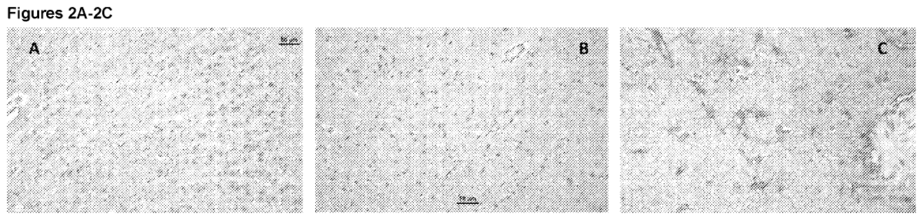

Infarction was not found in any brains on autopsy or on H&E staining. Slides for Pig #3 served as a control and was representative of the animals in the placebo group. Slides for Pigs #4 and #5 were representative of the DDFPe group. The H&E staining showed various neuronal morphological profiles (FIG. 1), which included Pig #4 (DDFPe) 1) near normal appearance of the parietal cortex (FIG. 1A) and dorsal hippocampus (FIG. 1D, damage score 0-1); Pig #5 (DDFPe) 2) scattered tissue edema and degenerated neurons in both cortex and hippocampus (FIG. 1B, 1E, damage score 2-3); and Pig #3 (placebo) 3) more severe ischemic damage with many neurons missing, few degenerated neurons (FIG. 1C, 1F, damage score 3-4). GFAP immunohistochemistry staining revealed progressively more activated astrocytes around the damaged area, and scattered gliosis in neuropils. (FIGS. 2A, 2B, and 2C) for Pigs #4, #5 and #3 respectively. Stages of injury noted in the animals are outlined in FIG. 5.

### Statistical Power Analysis

Assuming the probabilities of developing ROSC are 40% (=2/5) and 85.7% (=6/7) in the placebo and verum groups, respectively, the current study has a statistical power of 38.1 given sample sizes n=5 and 7. To achieve 80% statistical power in this test, the sample size required in each group is 16. Assuming the probabilities of survival are 20% (=1/5) and 57.1% (=4/7) in the placebo and verum groups, respectively, the current study has a statistical power of 17.3 given sample sizes n=5 and 7. To achieve 80% statistical power in this test, the sample size required in each group is 16.

### REFERENCES

[1] Tsao C W, Aday A W, Almarzooq Z I, Anderson C A M, Arora P, Avery C L, et al. Heart Disease and Stroke Statistics—2023 Update: A Report from the American Heart Association. vol. 147.:e93-e621, 2023.
[2] Sekhon M S, Ainslie P N, Griesdale D E. Clinical pathophysiology of hypoxic ischemic brain injury after cardiac arrest: a “two-hit” model. Crit Care 2017; 21(1):90.
[3] Benjamin E J, Blaha M J, Chiuve S E, Cushman M, Das S R, Deo R, et al. Heart Disease and Stroke Statistics-2017 Update: A Report From the American Heart Association. Circulation 2017; 135:e146-603.
[4] Valenzuela T D, Roe D J, Nichol G, Clark L L, Spaite D W, Hardman R G. Outcomes of Rapid Defibrillation by Security Officers After Cardiac Arrest in Casinos. N Engl J Med. 2000; 343:1206-9. ACC Curr J Rev 2001; 10:63-4.
[5] Hasselqvist-Ax I, Riva G, Herlitz J, Rosenqvist M, Hollenberg J, Nordberg P, et al. Early Cardiopulmonary Resuscitation in Out-of-Hospital Cardiac Arrest. N Engl J Med 2015; 372:2307-15.
[6] Olasveengen T M, de Caen A R, Mancini M E, Maconochie T K, Aickin R, Atkins D L, et al. 2017 International Consensus on Cardiopulmonary Resuscitation and Emergency Cardiovascular Care Science With Treatment Recommendations Summary. Resuscitation 2017; 121:201-14.
[7] Staffey K S, Dendi R, Brooks L A, Pretorius A M, Ackermann L W, Zamba K D, et al. Liquid ventilation with perfluorocarbons facilitates resumption of spontaneous circulation in a swine cardiac arrest model. Resuscitation 2008; 78:77-84.
[8] Chenoune M, Lidouren F, Adam C, Pons S, Darbera L, Bruneval P, et al. Ultrafast and whole-body cooling with total liquid ventilation induces favorable neurological and cardiac outcomes after cardiac arrest in rabbits. Circulation 2011; 124:901-7.
[9] Albaghdadi A S, Brooks L A, Pretorius A M, Kerber R E. Perfluorocarbon induced intra-arrest hypothermia does not improve survival in a swine model of asphyxial cardiac arrest. Resuscitation 2010; 81:353-8.
[10] Tyssebotn I M, Lundgren C E G, Olszowka A J, Guri W. Hypoxia Due to Shunts in Pig Lung Treated with O 2 and Fluorocarbon-derived Intravascular Microbubbles. Artif Cells, Blood Substitutes, Biotechnol 2010; 1199:79-89.
[11] Spiess B D. Perfluorocarbon Gas Transport: an Overview of Medical History With Yet Unrealized Potentials. Shock 2019; 52:7-12.
[12] Moon-Massat P, Abutarboush R, Pappas G, Haque A, Aligbe C, Arnaud F, et al. Effects of Perfluorocarbon Dodecafluoropentane (NVX-108) on Cerebral Microvasculature in the Healthy Rat. Curr Drug Discov Technol 2014; 11:220-6.
[13] Arthur C, Song L, Culp W, Brown A, Borrelli M, Skinner R, et al. Tissue Concentration of Dodecafluoropentane (DDFP) Following Repeated I V Administration in the New Zealand White Rabbit. AAPS J 19, 520-526 (2017).
[14] Mullah S H, Saha B K, Abutarboush R, Walker P B, Haque A, Arnaud F G, et al. Perfluorocarbon NVX-108 increased cerebral oxygen tension after traumatic brain injury in rats. Brain Res 2016; 1634:132-9.
[15] Swyer T W, Strom J, Larson D F. Nanoparticle oxygen delivery to the ischemic heart. Perfus (United Kingdom) 2014; 29:539-43.
[16] Culp W C, Onteddu S S, Brown A, Nalleballe K, Sharma R, Skinner R D, et al. Dodecafluoropentane Emulsion in Acute Ischemic Stroke: A Phase Ib/II Randomized and Controlled Dose-Escalation Trial. J Vasc Interv Radiol 2019. 30 (8) 1244-1250. E1
[17] Spiess B D. Perfluorocarbon emulsions as a promising technology: a review of tissue and vascular gas dynamics. J Appl Physiol 2009; 106:1444-52.
[18] Wall T C, Califf R M, Blankenship J, Talley J D, Tannenbaum M, Schwaiger M, et al. Intravenous Fluosol in the treatment of acute myocardial infarction. Results of the Thrombolysis and Angioplasty in Myocardial Infarction 9 Trial. TAMI 9 Research Group. Circulation 1994; 90:114-20.
[19] Spahn D R. Artificial oxygen carriers: a new future?Crit Care 2018; 22:46.
[20] Brown A T, Arthur M C, Nix J S, Montgomery J A, Skinner R D, Roberson P K, et al. Dodecafluoropentane Emulsion (DDFPe) Decreases Stroke Size and Improves Neurological Scores in a Permanent Occlusion Rat Stroke Model. Open Neurol J 2014. 8, 27-33.
[21] Johnson J L H, Dolezal M C, Kerschen A, Matsunaga T O, Unger E C. In vitro comparison of dodecafluoropentane (DDFP), perfluorodecalin (PFD), and perfluoroctylbromide (PFOB) in the facilitation of oxygen exchange. Artif Cells, Blood Substitutes, Biotechnol 2009; 37:156-62.
[22] Culp W C, Woods S D, Skinner R D, Brown A T, Lowery J D, Johnson J L H, et al. Dodecafluoropentane emulsion decreases infarct volume in a rabbit ischemic stroke model. J Vasc Interv Radiol 2012 23 (1) 116-121.
[23] Woods S D, Skinner R D, Brown A T, Ricca A M, Johnson J L, Unger E C, et al. Repeated doses of dodecafluoropentane emulsion provide neuroprotection up to 24 hours following cerebral artery occlusion in rabbits. Stroke 2013; 44:A190
[24] Liu Z, Barber C, Gupta A, Wan L, Won Y W, Furenlid L R, et al. Imaging assessment of cardioprotection mediated by a dodecafluoropentane oxygen-carrier administered during myocardial infarction. Nucl Med Biol 2019. Volume 70, Pages 67-77.
[25] Correas J-M, Kessler D R, Worah D I, Quay S C. Ultrasound Contrast Agent Evaluated with Gas. Medicine (Baltimore) 2001; 27:565-70.
[26] Quay S, Eisenfeld A. Safety Assessment of the Use of Perflenapent Emulsion for Contrast Enhancement of Echocardiography and Diagnostic Radiology Ultrasound Studies. Clin Cardiol 1997; 20:119-26.
[27] Graham K, Moon-Massat P F, Unger E C. Dodecafluoropentane Emulsion (DDFPE) as a Resuscitation Fluid for Treatment of Hemorrhagic Shock and Traumatic Brain Injury: A Review. Shock 2019; October; 52 (iS Suppl; 50-54.
[28] Strom J, Swyers T, Wilson D, Unger E, Chen Q M, Larson D F. Dodecafluoropentane emulsion elicits cardiac protection against myocardial infarction through an ATP-Sensitive K+ channel dependent mechanism. Cardiovasc Drugs Ther 2014; 28:541-7.
[29] Babini G, Ristagno G, Boccardo A, De Giorgio D, De Maglie M, Affatato R, et al. Effect of mild hypercapnia on outcome and histological injury in a porcine post cardiac arrest model. Resuscitation 2019; 135:110-7.
[30] Wenzel V, Lindner K H, Krismer A C, Voelckel W G, Schocke M F, Hund W, et al. Survival with full neurologic recovery and no cerebral pathology after prolonged cardiopulmonary resuscitation with vasopressin in pigs. J Am Coll Cardiol 2000; 35:527-33.
[31] Tang W, Weil M H, Sun S, Povoas H P, Klouche K, Kamohara T, et al. A Comparison of Biphasic and Monophasic Waveform Defibrillation After Prolonged Ventricular Fibrillation. Chest 2001; 120:948-54.
[32] Xanthos T, Bassiakou E, Koudouna E, Rokas G, Goulas S, Dontas I, et al. Combination pharmacotherapy in the treatment of experimental cardiac arrest. Am J Emerg Med 2009; 27:651-9.
[33] Janata A, Weihs W, Schratter A, Bayegan K, Holzer M, Frossard M, et al. Cold aortic flush and chest compressions enable good neurologic outcome after 15 mins of ventricular fibrillation in cardiac arrest in pigs. Crit Care Med 2010; 38:1637-43.
[34] Janata A, Bayegan K, Weihs W, Schratter A, Holzer M, Frossard M, et al. Emergency preservation and resuscitation improve survival after 15 minutes of normovolemic cardiac arrest in pigs. Crit Care Med 2007; 35:2785-91.
[35] Elmelige Y O, Yang Z, Chowdhury M A B, Gul S S, Cohen S A, Youn T S, et al. Neural biomarker level after out-of-hospital cardiac arrest predicts neurological outcome. Acad Emerg Med 2019; 26:S2019 Annual; Meeting Supplement: S30.
[36] Gul S S, Huesgen K W, Wang K K, Mark K, Tyndall J A. Prognostic utility of neuroinjury biomarkers in post out-of-hospital cardiac arrest (OHCA) patient management. Med Hypotheses 2017 105:34-47.
[37] Murphy T W, Cueto R, Zhu J, Spiess B, Eurell L B, Becker T K. Ultrasound-guided external jugular and femoral arterial cannulation for juvenile swine. Lab Anim 2021; 55:573-6.
[38] Babini G, Grassi L, Russo I, Novelli D, Boccardo A, Luciani A, et al. Duration of Untreated Cardiac Arrest and Clinical Relevance of Animal Experiments. SHOCK 2018; 49:205-12.
[39] R Core Team. R: A language and environment for statistical computing. Vienna, Austria: R Foundation for Statistical Computing; 2021.
[40] Bates D, Machler M, Bolker B M, Walker S C. Fitting linear mixed-effects models using lme4. J Stat Softw 2015; 67 (1) 1-48.
[41] H W. ggplot2: Elegant Graphics for Data Analysis. New York: Springer-Verlag; 2016.
[42] Wada T. Coagulofibrinolytic changes in patients with post-cardiac arrest syndrome. Front Med 2017; 4:1-11.
[43] Krupickova P, Mormanovi Z, Boucek T, Belza T, Smalcova J, Belohlivek J. Microvascular perfusion in cardiac arrest: a review of microcirculatory imaging studies. Perfusion. 2018; 33(1):8-15.
[44] Li L, Poloyac S M, Watkins S C, Croix C M St., Alexander H, Gibson G A, et al. Cerebral microcirculatory alterations and the no-reflow phenomenon in vivo after experimental pediatric cardiac arrest. Journal of Cerebral Blood Flow & Metabolism. 2019; 39(5):913-925.
[45] Quay S C, Eisenfeld A J. Safety Assessment of the Use of Pefflenapent Emulsion for Contrast Enhancement of Echocardiography and Diagnostic Radiology Ultrasound Studies. Clin Cardiol 1997; 20:119-26.
[46] Jayaraman M S, Graham K, Unger E C. In vitro model to compare the oxygen offloading behaviour of dodecafluoropentane emulsion (DDFPe). Artif Cells, Nanomedicine Biotechnol 2019. 47:1, 783-789.
[47] Fries M, Weil M H, Chang Y Te, Castillo C, Tang W. Microcirculation during cardiac arrest and resuscitation. Crit Care Med 2006; 34:2-5.
[48] Spahn D R, van Brempt R, Theilmeier G, Reibold J-P, Welte M, Heinzerling H, et al. Perflubron Emulsion Delays Blood Transfusions in Orthopedic Surgery. Anesthesiology 1999; 91:1195.
[49] Grayburn P A, Weiss J L, Hack T C, Klodas E, Raichlen J S, Vannan M A, et al. Phase III multicenter trial comparing the efficacy of 2% dodecafluoropentane emulsion (EchoGen) and sonicated 5% human albumin (Albunex) as ultrasound contrast agents in patients with suboptimal echocardiograms. J Am Coll Cardiol 1998; 32:230-6.
[50] Jover, T. Tanaka H., Calderone A, et al. Estrogen protects against global ischemia-induced neuronal death and prevents activation of apoptotic signaling cascades in the hippocampal CA1. J. Neurosci. 2002; 22(6):2115-2124.
[51] Culp W C, et al., Mol Neurobiol 2015; 52:979-984
[52] Arthur M C, et al., Mol Neurobiol 2017; 54:4764-4770
[53] Woods S D, et al., Mol Neurobiol 2013; 48:363-367
[54] Deuchar G A, et al., Theranostics 2018; 8: 1706-1722
[55] Grayburn P A, et al., J Am Coll Cardiol 1995; 26: 1340-1347
[56] The European Agency for the Evaluation of Medicinal Products Human Medicines Evaluation Unit, Jul. 27, 1998, CPMP/1342/98.
[57] The European Agency for the Evaluation of Medicinal Products Human Medicines Evaluation Unit, London, May 22, 2001, Doc. Ref: EMEA/37043/00).
[58] Grayburn, P. Clin. Cardiol. Vol. 20 (Suppl. I), 1-12-1-18 (1997).
[59] Worah, D. M. et al., Drugs of the Future 1997 22 (4) 378-385).
[60] EchoGen Package Leaflet, EMEA/H/C/000149 Annex I, Summary of Product Characteristics, anx_3235
[61] Lundgren, C. et al., UHM 2005, Vol. 32, No. 4, p.215-226
[62] Lundgren, C. E. G. et al., Artificial Cells, Blood Substitutes, and Biotechnology, 34:473-486 (2006)
[63] Johnson, J. L. H. et al. J. Biomed. Nanotechnol. 2015 February; 11(2):274-281.
[64] Arnaud, F. et al., Med. Sci. 2020, 8, 41
[65] Moseby-Knappe, M., et al., Intensive Care Med. Intensive Care Med. 2022 February; 48(2):248
[66] Berg R A, et al., Crit Care Med, 1994; 22(2): 282-290
[67] Tang W, et al.,. Circulation, 1997; 95(5): 1335-1340
[68] Keipert, P., Artif Cells Blood Substit Immobil Biotechnol 1995; 23(3):381-94
[69] Flaim S. F. et al., Artif Cells Blood Substit Immobil Biotechnol 1994; 22(4):1511-5).

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, patents, and patent applications cited in this specification are herein incorporated by reference as if each was specifically and individually indicated to be incorporated by reference.

1. A method of treating a subject with cardiac arrest, comprising administering to the subject a pharmaceutical composition comprising NANO2 dodecafluoropentane emulsion (NuvOx Pharma, LLC, Tucson, Arizona).

2. The method of claim 1, wherein the cardiac arrest is associated with coronary artery disease, valvular heart disease, cardiac dysrhythmias, myocardial infarction, pulmonary disease, arrhythmia, stroke, shock, sepsis, pneumonia, pulmonary embolism, trauma, or COVID-19.

3. The method of claim 1, wherein blood flow has stopped in the subject.

4. The method of claim 1, wherein the pharmaceutical composition is administered intravenously.

5. The method of claim 1, wherein the pharmaceutical composition is injected intravenously via bolus over about 1 to about 10 minutes at a dose of about 0.3 mL/kg of subject body weight to about 1 mL/kg of subject body weight.

6. The method of claim 1, wherein the pharmaceutical composition is injected intravenously via slow IV push over about 1 to about 15 minutes at a dose of about 0.3 mL/kg to about 1 mL/kg of subject body weight.

7. The method of claim 6, wherein the pharmaceutical composition is injected intravenously via slow IV push over about 1 to about 10 minutes.

8. The method of claim 6, wherein the pharmaceutical composition is injected intravenously via slow IV push over about 5 to about 15 minutes.

9. The method of claim 4, wherein the pharmaceutical composition is injected intravenously at a dose of about 0.5 mL/kg of subject body weight.

10. The method of claim 9, wherein the pharmaceutical composition is injected intravenously over about 10 minutes at a dose of about 0.5 mL/kg of subject body weight.

11. The method of claim 1, wherein the pharmaceutical composition is administered as an IV infusion at a rate of about 0.05 mL/kg of subject body weight per hour to about 0.15 mL/kg of subject body weight per hour.

12. The method of claim 1, wherein the pharmaceutical composition is injected intravenously as a sustained IV infusion at a rate of about 0.001 mL/kg of subject body weight per hour to about 0.015 mL/kg of subject body weight per hour.

13. The method of claim 1, wherein the subject is a mammal.

14. The method of claim 13, wherein the mammal is a primate.

15. The method of claim 13, wherein the mammal is a non-human primate.

16. The method of claim 13, wherein the mammal is a human.

17. The method of claim 13, wherein the mammal is a rodent.

18. The method of claim 17, wherein the rodent is a mouse, rat, guinea pig, hamster, or gerbil.

19. The method of claim 13, wherein the mammal is selected from the group consisting of human, baboon, chimpanzee, monkey, cynomolgus, marmoset, rhesus, rodent, rabbit, cat, dog, horse, cow, sheep, goat, pig, ferret, guinea pig, hamster, and gerbil.
